(19) United States
(12) Patent Application Publication
(10) Pub. No.: US 2005/0142641 A1
(43) Pub. Date: Jun. 30, 2005

(54) Group I mite polypeptide variants

(75) Inventors: Nanna Kristensen Soni, Copenhagen O (DK); Esben Peter Friis, Valby (DK); Erwin Ludo Roggen, Lyngby (DK); Stina Thulesen Lyngstrand, Horsholm (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/831,027

(22) Filed: Apr. 23, 2004

Related U.S. Application Data
(60) Provisional application No. 60/466,802, filed on Apr. 30, 2003.
(60) Provisional application No. 60/490,867, filed on Jul. 29, 2003.

(30) Foreign Application Priority Data
Apr. 25, 2003 (DK) PA 2003 00628
Jul. 21, 2003 (DK) PA 2003 01093

(51) Int. Cl. C07K 14/435; C07H 21/04; C12N 5/06

(52) U.S. Cl. 435/69.3; 435/348; 435/320.1; 530/395; 536/23.5

(57) ABSTRACT

This invention concerns variants of group 1 mite polypeptides, wherein the mature polypeptide of the variant comprise one or more mutations in the positions or corresponding to the positions consisting of A10, A12, E13, G29, G30, G32, A46, Y47, S54, L55, D64, A66, S67, G73, T75, I80, Q84, N86, G87, S92, Y93, Y96, A98, R99, E100, Q101, R104, R105, P106, Q109, R110, F111, G112, I113, A132, I144, K145, D146, D148, R151, I158, I159, Q160, R161, D162, N163, G164, Y165, Q166, N179, A180, G182, V183, D184, A205, I208 of SEQ ID NO: 1 or 10, 12, 13, 29, 30, 32, 46, 47, 54, 55, 64, 66, 67, 73, 75, I80, 84, 86, 87, 92, Y93, 96, 98, 99, 100, 101, 104, 105, 106, 109, 110, 111, 112, 113, 132, 144, 145, 146, 148, 151, 158, 159, 160, 161, 162, 163, 164, 165, 166, 179, 180, 182, 183, 184, 205, 208 of the mature Der p 1 polypeptide.

### CROSS-REFERENCE TO RELATED APPLICATIONS

This application claims priority or the benefit under 35 U.S.C. 119 of Danish application nos. PA 2003 00628 and PA 2003 01093 filed Apr. 25, 2003 and Jul. 21, 2003 and U.S. provisional application Nos. 60/466,802 and 60/490,867 filed Apr. 30, 2003 and Jul. 29, 2003, the contents of which are fully incorporated herein by reference.

### REFERENCE TO A SEQUENCE LISTING

The present application contains information in the form of a sequence listing, which is appended to the application and also submitted on a data carrier accompanying this application. The contents of the data carrier are fully incorporated herein by reference.

### BACKGROUND OF THE INVENTION

1. Field of the Invention

The present invention relates to variants of the group 1 mite polypeptide antigens allergens having an altered antigenic profile, compared to the parent group 1 polypeptide allergens, processes for making such variants, compositions comprising the variants and use of the variants in immuno-therapy such as allergy vaccination and/or desensitisation.

2. Description of Related Art

Antigenic polypeptides heterologeous to humans and animals, such as the group 1 mite polypeptide allergens, present e.g., in excrements of dust mites Dermatophagoides pteronyssinus (Der p 1) or Dermatophagoides farinae (Der f 1), can induce immunological responses in susceptible individuals, such as an atopic allergic response, in humans and animals. Allergic responses may range from hay fever, rhinoconjunctivitis, rhinitis, and asthma, and in cases when the sensitised individual is exposed, e.g., to bee sting or insect bites, even to systemic anaphylaxis and death.

An individual may become sensitised to such polypeptides, termed allergens, by inhalation, direct contact with skin or eyes, ingestion or injection. The general mechanism behind an allergic response is divided into a sensitisation phase and a symptomatic phase. The sensitisation phase involves a first exposure of an individual to an allergen. This event activates specific T- and B-lymphocytes, and leads to the production of allergen specific antibodies, such as immunoglobulin E (IgE). The specific IgE antibodies bind to IgE receptors on mast cells and basophils, among others, and the symptomatic phase is initiated upon a second exposure to the same or a homologous allergen. The allergen will bind to the cell-bound IgE, and the polyclonal nature of the antibodies results in bridging and clustering of the IgE receptors, and subsequently in the activation of mast cells and basophils. This activation results in the release of various chemical mediators, such as histamine, heparin, proteases, prostaglandin D2 and leukotrienes, involved in the early as well as late phase reactions of the symptomatic phase of allergy.

For certain forms of IgE-mediated allergies, a therapy exists, called specific allergy vaccination (SAV) or immuno therapy (IT), which comprises repeated parenteral or mucosal (e.g., sublingual) administration of allergen preparations formulated as a vaccine (Int. Arch. Allergy Immunol., 1999, vol. 119, pp 1-5). This leads to reduction of the allergic symptoms, most likely due to induction of a protective, non IgE-based immune response, possibly by modulation of the existing Th2 response and/or a redirection of the immune response towards the immunoprotective (Th1) pathway (Int. Arch. Allergy Immunol., 1999, vol. 119, pp 1-5).

Compared to other types of vaccination, allergy vaccination is complicated by the presence of an existing and ongoing immune response in the allergic patients. The presence of allergen specific IgE antibodies on effector cells, such as mast cells and basophils, in affected tissues, may result in allergic symptoms upon exposure to antigens. Thus, the inherent risk of adverse events or side effects limits the antigen dose, which can be administered, and has necessitated prolonged (12-36 months) and cumbersome treatment regimes where the delivered dose slowly is increased over time.

There is thus a need to provide modified allergens, with a lower inherent risk of inducing adverse events, which can be used for specific allergy vaccination. These modified allergens should have a reduced capacity for binding, and especially cross-linking, antigen-specific IgE molecules. At the same time it is important that they retain the tertiary structure, and to some degree the immunogenicity, of the parent allergen, in order to be able to elicit the protective (IgG-based) immune response in the patient.

In order to produce such modified proteins it is desirable to first identify the minimal B cell epitopes on the molecule. An epitope is the structural area on a complex antigen that can combine with an antibody, while the minimal epitope contains the amino acids involved directly in antibody binding.

B-cell epitopes can in nature be continuous, discontinuous or a combination thereof, but must contain around 10 amino acids in order to elicit an antibody response. One may identify larger regions or areas of the molecule comprising an epitope and a minimal epitope, but when desiring to alter immunogenic properties of a polypeptide by introducing mutations in the molecule one will realize the importance of firstly identifying the minimal epitope because it is far less feasible to prepare modified polypeptides by mutating amino acids if the number of amino acids, which potentially is to be mutated, exceeds 5-10 amino acids. This is because the number of possible variants increases steeply with the number of amino acids involved in the mutation strategy (many more permutations possible) and because it may be less useful to modify amino acids which are not part of an epitope.

Several studies have been aimed at identifying epitopes in group 1 dust mite allergens:

Green et al., Int. Arch. Allergy Appl. Immunol., vol. 92, pp 30-38, 1990; Green et al. J. Immunol., vol. 147 pp. 3768-3773, 1991 and Green & Thomas, Mol. Immunol. Vol. 29(2), pp 257-262, 1992, disclose antibody-binding fragments of Der p 1. The antibody binding regions disclosed are very large (from 11-56 amino acid residues) and together they cover almost all amino acids of the molecule.

Lombardero et al., J. Immunol., vol. 144(4), pp 1353-1360, 1990, disclose that B cell epitopes on Der p 1 are conformational, i.e., the epitope is made up of non-contiguous parts of the molecule and thus highly dependent on correct tertiary structure, and that antibody binding is sensitive to denaturation of the protein.

Collins et al., Clin. Exp. Allergy, vol. 26(1), pp 36-42, 1996, conclude that IgE binding epitopes of Der p 1 and Der f 1 are discontinuous in nature.

Jeannin et al., Mol. Immunol. Vol. 29(6), pp 739-749, 1992, used predictions of hydrophobicity and solvent accessibility to amino acid residues on a three-dimensional model of Der p 1 to identify 4 putative antibody binding peptides: N52-C71, C117-Q133, G176-I187 and V188-Y199. The four peptides could induce low levels of histamine release in basophils from 40-60% of a panel of dust mite allergic patients. Histamine release requires cross-linking of at least two IgE molecules and the authors speculate that the peptides must have bound non-specifically to serum components, and thus acted as haptens.

Furmonaviciene et al., Clin. Exp. Allergy, 29, pp 1563-1571, 1999, suggest L147 to Q160 of Der p 1 to be the potential epitope recognised by a monoclonal mouse anti-Der p 1 antibody.

Pierson-Mullany et al., Mol. Immunol., 37, pp 613-620, 2000, reported that peptides representing residues T1 to T21, E59 to Y93, Y155 to W187 and I209 to I221 of Der p 1 can parially inhibit human serum binding to Der p 1.

WO 99/47680 (ALK-ABELLÓ) discloses that allergens may be modified to render these polypeptides less allergenic. This disclosure concerns mainly modification of the birch pollen protein, Bet v 1 and Venom allergen Ves V 5.

WO 02/40676 (ALK-ABELLÓ) discloses modified allergens, said modifications allegedly causing the allergenicity of the allergen to be reduced. In this disclosure amino acids suitable for modification are selected by virtue of their solvent accessibility, i.e. if they are present on the surface of the allergen or they are selected if they are conserved vis a vis homologeous allergens of the same taxonomic group.

WO 01/29078 (HESKA) describes recombinant expression of group 1 mite proteins, nucleotide sequences encoding these proteins and nucleotide sequences modified to enable expression of the proteins in certain microorganisms. The group 1 mite polypeptides of this disclosure are said to bind to IgE which also bind to native group 1 mite polypeptides.

EP-A-1 219 300 describes a method for administering an allergy vaccine.

In the art various suggestions are made as to the antibody binding epitopes of group 1 mite polypeptides such as Der p 1. However, the art either concerns (1) linear epitopes of which most have low relevance in allergy, (2) regions which are too large to contain information of specific amino acids involved in antibody recognition, (3) epitopes selected by choosing those areas which have a higher solvent accessibility without considering if the epitope is de facto involved in antibody binding, (4) non human epitopes or (5) a combination of one or more of (1)-(4).

The inadequacy of the epitope identification in for example Der p 1 may be the reason why very different potential epitopes on for example Der p 1 have been reported in different documents. For example in WO 02/40676 residues E13, P24, R20, Y50, S67, R78, R99, Q109, R128, R156, R161, P167 and W192 are selected as being important for the allergenicity of Der p 1, while in Pierson-Mullany et al. the epitopes are contemplated to be T1 to T21, E59 to Y93, Y155 to W187 and I209 to I221, in Furmonaviciene et al. the major epitope is determined to be L147 to Q160, and in Jeannin et al, (where an almost identical approach as in WO 02/40676 is utilised), N52-C71, C117-Q133, G176-1187 and V188-Y199 are identified.

Further, two studies have aimed to reduce activity or ‘allergenic activity’ by mutation in residues of the active site, maturation site, or cysteine-bridge formation sites:

WO 99/25823 (Smith Kline Beecham) discloses variants of Der p 1 in which a) C34 is mutated, b) the pro-peptide site is modified, e.g. by deletion of NAET sequence or c) H170 is mutated.

WO 03/016340 (Smith Kline Beecham) discloses variants of Der p 1 in which either of six cysteinses (C4, C31, C65, C71, C103, or C117) are mutated.

The ambiguity of the art concerning epitopes, of e.g., Der p 1, means that presently no conclusive and reliable data is available on epitopes of Group 1 mite polypeptides and even less on amino acids comprised in said epitopes suitable for mutation with the purpose of reducing the antigenicity of these polypeptides.

### SUMMARY OF THE INVENTION

Attempts to reduce the allergenicity of polypeptide allergens have been conducted. It was found that small changes in the epitope, may affect the binding to an antibody. This may change the properties of such an epitope, e.g., by converting it from a high affinity to a low affinity epitope towards antibodies, or even result in epitope loss, i.e. that the epitope cannot sufficiently bind an antibody to elicit an antigenic response.

In order to produce modified group 1 mite polypeptides with improved properties as a vaccine agent, it is an advantage to first identify the minimal B cell epitopes on the molecule. An epitope is the smallest structural area on a complex antigen that can bind an antibody. B-cell epitopes can be continuous or discontinuous in nature. The minimal epitope consists of the specific amino acids directly involved in antibody binding.

The present invention relates to variants of group 1 mite polypeptide antigens, including Der p 1, comprising a mutation in a minimal epitope and thus having an altered immunogenic profile in exposed animals, including humans.

The applicant has identified amino acids in group 1 mite polypeptides which are involved in antibody binding epitopes and for which a mutation have an altering, preferably reducing, effect on the antibody binding, particularly IgE binding of the polypeptide. Hence, the present invention provide in a first aspect a variant of a group 1 mite polypeptide, wherein the mature polypeptide of the variant comprises one or more mutations in the positions or corresponding to the positions consisting of A10, A12, E13, G29, G30, G32, A46, Y47, S54, L55, D64, A66, S67, G73, T75, I80, Q84, N86, G87, S92, Y93, Y96, A98-Q101, R104-P106, Q109-I113, A132, I144-D146, D148, R151, I158-Q166, N179, A180, G182-D184, A205, I208 of SEQ ID NO: 1 or alternatively 10, 12, 13, 29, 30, 32, 46, 47, 54, 55, 64, 66, 67, 73, 75, 80, 84, 86, 87, 92, 93, 96, 98-101, 104-106, 109-113, 132, 144-146, 148, 151, 158-166, 179-180, 182-184, 205, 208 of the mature Der p 1 polypeptide.

Particularly, said variants have reduced IgE-binding, more particularly combined with preserved immunogenicity for inducing protective responses (vide supra). Still more preferably, the variants have an altered immunogenic profile in exposed animals, including humans, as compared to the native group 1 mite polypeptide.

In further aspects the invention provides a nucleotide sequence encoding the variant of the invention; a nucleotide construct comprising the nucleotide sequence encoding the variant, operably linked to one or more control sequences that direct the production of the variant in a host cell; a recombinant expression vector comprising the nucleotide construct of the invention and to a recombinant host cell comprising the nucleotide construct of the invention.

In a still further aspect the invention provides a method of preparing a variant of the invention comprising:
(a) cultivating a recombinant host cell of the invention under conditions conducive for production of the variant of the invention and
(b) recovering the variant.

In still further aspects the invention provides a composition comprising a variant of the invention and a pharmaceutically acceptable carrier and a method for preparing such a pharmaceutical composition comprising admixing the variant of the invention with an acceptable pharmaceutical carrier.

In still further aspect the invention provides a variant or a composition of the invention for use as a medicament.

In still further aspect the invention provides use of a variant or the composition of the invention for the preparation of a medicament for the treatment of an immunological disorder.

In still further aspect the invention provides use of the variant or the composition of the invention for the treatment of a disease.

In still further aspect the invention provides use of the variant or the composition of the invention for the treatment of an immunological disorder.

In a still further aspect the invention provides a kit comprising the variant of the invention immobilized on a solid support.

### BRIEF DESCRIPTION OF THE DRAWINGS

FIG. 1 shows histamine release in one representative donor, donor 1, in response to stimulation with group 1 mite polypeptide (nDerp1) and the group 1 mite polypeptide variants, rec-proDer p 1, rec-Der p 1 and DP070.

FIG. 2 shows normalized histamine release, EC₅₀ was calculated for the group 1 mite polypeptide variants. The dose response curves in nDer p 1-specific IgE serum isolated from 14 patients with dust-mite allergy were plotted and fitted to a sigmoid curve, and the EC₅₀ was calculated for group 1 mite polypeptide variants and normalized to group 1 mite polypeptide (nDer p 1).

### DETAILED DESCRIPTION OF THE INVENTION

Definitions

The term “native polypeptide” as used herein is to be understood as a polypeptide essentially in its naturally occurring form. A native polypeptide may be for example be a wild type polypeptide, i.e. a polypeptide isolated from the natural source, a polypeptide in its naturally occurring form obtained via genetic engineering by expression in host organism different from the natural source or by polypeptide synthesis.

An “epitope” or a “B-cell epitope”, as used in this context, is an antigenic determinant and the structural area on a complex antigen that can combine with or bind an antibody. It can be discontinuous in nature, but will in general have a size of 1 kD or less (about 10 amino acids or less). The size may be 3 to 10 amino acids or 5 to 10 amino acids or even 7 to 10 amino acids, depending on the epitope and the polypeptide.

The term “epitope pattern” as used herein is to be understood as a consensus sequence of antibody binding peptides. An example is the epitope pattern A R R * R. The sign “*” in this notation indicates that the aligned antibody binding peptides included a non-consensus moiety between the second and the third arginine. That moiety may be any amino acid or a few amino acids or no amino acid. Epitope patterns are used to identify epitopes and minimal epitopes on complex antigens.

The term “Anchor amino acid” as used herein is to be understood as conserved individual amino acids of an epitope pattern recurring in all peptides bound by monospecific antibodies used to define that pattern. Anchor amino acid will usually also be the amino acid of a minimal epitope on the full polypeptide.

The “antigenicity” of a polypeptide indicates, in this context, its ability to bind antibodies e.g., of IgE and/or IgG and/or other immunoglobulin classes. The ‘IgE-antigenicity’ of a polypeptide as used herein, indicates its ability to bind IgE antibodies.

The “immunogenicity” of a polypeptide indicates its ability to stimulate antibody production and immunological reactions in exposed animals, including humans.

The “allergenicity” of a polypeptide indicates its ability to stimulate IgE antibody production and allergic sensitization in exposed animals, including humans.

The term “parent” or “parent group 1 mite polypeptide” is to be understood as a group 1 mite polypeptide (also refered to as group 1 mite allergen) before introducing the mutations according to the invention. In particular the parent group 1 mite polypeptide is the native group 1 mite polypeptide.

Group 1 Mite Polypeptides

As described in the art such as WO 01/29078, mites produce several classes or groups of allergens, one of which is known as Group 1 allergens. Group 1 allergens, displaying considerable cross-reactivity, have been found in Dermatophagoides pteronyssinus, Dermatophagoides farinae, Dermatophagoides siboney, Dermatophagoides microceaus, Blomia tropicalis and Euroglyphus maynei, see for example, Thomas et al, 1998, Allergy 53, 821-832.

Group 1 mite allergens share significant homology with a family of cysteine proteases including actinidin, papain, cathepsin H and cathepsin B, which is why they often are referred to as Group 1 mite cysteine proteases. The Group 1 mite allergens are commonly found in the feces of mites and are thought to function as digestive enzymes in the mite intestine.

Group 1 allergens from different mites are highly homologous, approximately 25 kilodalton (kD) secretory glycoproteins, that are synthesized by the cell as a pre-pro-protein that is processed to a mature form. D. farinae, D. pteronyssinus, and E. maynei Group 1 proteins, for example, share about 80% identity. In particular, Group 1 allergens from D. farinae and D. pteronyssinus, also referred to as Der f 1 and Der p 1 proteins, respectively, show extensive cross-reactivity in binding IgE and IgG. In patients that are mite allergic, approximately 80% to 90% of the individuals have IgE that is reactive to Group 1 allergens (Thomas, Adv. Exp. Med. Biol., 409, pp. 85-93, 1996).

Group 1 mite allergens thus include native polypeptides known in the art as Der p 1 obtainable from Dermatophagoides pteronyssinus (NCBI accession number: P08176, SEQ ID NO: 1), Der f 1 obtainable from Dermatophagoides farinae (NCBI accession number: P16311, SEQ ID NO: 2), Eur m 1 obtainable from Euroglyphus maynei (NCBI accession number: P25780, SEQ ID NO: 3), Der m 1 obtainable from Dermatophagoides microceaus (NCBI accession number: P16312, SEQ ID NO: 4), and Blo t 1 obtainable from Blomia tropicalis (NCBI accession number: Q95PJ4, SEQ ID NO: 5). Thus, in the context of this patent, the term group 1 mite allergens includes in particular native group 1 mite allergens, but also includes homologs to the native group 1 allergens, such as recombinant variants with disrupted N-glycosylation motifs, and hybrids of the above mentioned mite allergens, e.g. as created by family shuffling as described in the art (J. E. Ness, et al, Nature Biotechnology, Vol. 17, pp. 893-896, 1999).

In Silico Identification of Epitope Patterns and Epitopes in Group 1 Mite Polypeptides

Group 1 mite polypeptides may be epitope mapped using the proprietary in silico epitope mapping tool disclosed in detail in WO 00/26230 and WO 01/83559. In brief, this tool comprises a database of epitope patterns (determined from an input of peptide sequences, known to bind specifically to anti-protein antibodies) and an algorithm to analyse 3-D structure of a given protein against the epitope pattern database. This will determine the possible epitopes on that protein, and the preference of each amino acid in the protein sequence to be part of epitopes.

Identifying Antibody-Binding Peptides:

Antibody-binding peptides can be identified by many different ways. One is to synthesize a number of peptides of known sequence, and test for their ability to bind antibodies of interest, e.g., in ELISA or other immunochemical assays. Such data are available in great abundance in the literature.

A particularly effective way is to prepare a library of many different random peptide sequences and select experimentally only the ones that bind antibodies well and specific (i.e., can be outcompeted by the protein towards which the antibodies were raised). Phage display techniques are well suited for this way of finding antibody bidning peptides:

In a phage display system, a sequence encoding a desired amino acid sequence is incorporated into a phage gene coding for a protein displayed on the surface of the phage. Thus, the phage will make and display the hybrid protein on its surface, where it can interact with specific target agents. Given that each phage contains codons for one specific sequence of a determined length, an average phage display library can express 10⁸-10¹² different random sequences. If the displayed sequence resembles an epitope, the phage can be selected by an epitope-specific antibody. Thus, it is possible to select specific phages from the bulk of a large number of phages, each expressing their one hybrid protein.

It is important that the amino acid sequence of the (oligo)peptides presented by the phage display system have a sufficient length to present a significant part of an epitope to be identified. The oligopeptides may have from 5 to 25 amino acids, preferably at least 8 amino acids, such as 9 amino acids.

The antibodies used for reacting with the oligopeptides can be polyclonal or monoclonal. In particular, they may be IgE antibodies to ensure that the epitopes identified are IgE epitopes, i.e., epitopes inducing and binding IgE. The antibodies may also be monospecific, meaning they are isolated according to their specificity for a certain protein. Polyclonal antibodies are preferred for building up data on antibody-binding peptides to be used in the in silico mapping tool in order to obtain a broader knowledge about the epitopes of a polypeptide.

These reactive peptides, by virtue of their reactivity against antibodies, to some degree resemble the appearance of an epitope on a full polypeptide.

Identifying Epitope Patterns from Reactive Petides

The reactive (oligo)peptides identified e.g. by phage display are compared and aligned in order to identify common epitope patterns, which then can be used for identification of antibody binding epitopes on a 3-dimensional polypeptide.

In the alignment conservative alternatives to an amino acid such as aspartate and glutamate, lysine and arginine, serine and threonine are considered as one or equal.

Thus, the alignment results in a number of patterns, which depend on the chosen number of residues of the peptides. Using for example a 7-mer peptide, the pattern may have the form:
X X * * X X X,
   where “*” in this notation indicates a non-consensus moiety which may be any amino acid or group of amino acids or no amino acid, while X is one of the following 13 residue types: AG, C, DE, FY, H, IL, KR, M, NQ, P, ST, V, and W, where the pairs AG, DE, FY, IL, KR, NQ, ST are conservative alternatives and considered equal. Accordingly, 3 peptides such as
A K S N N K R
A K S M N K R
A K T P N K K
   would create a pattern of [AG] [KR] [ST] * [NQ] [KR] [KR], where the residues AG KR ST and NQ KR KR are consensus residues shared by all 3 peptides and thus the epitope pattern would be AG KR ST * NQ KR KR. The patterns are chosen to describe a complete set of reactive (oligo)peptides (obtained e.g., by a phage display and antibody reaction) by the fewest possible patterns.

The epitope patterns may be determined directly from the reactive peptides; if for example a library of 7-mer reactive peptides is made, one can use each different reactive 7 mer peptide, taking conservative alternatives into account, as an epitope pattern in the epitope mapping approach as described below.

It is also possible to reduce the number of epitope patterns to be examined in the epitope mapping by removing redundant patterns and/or by employing experimental designs as known in the art (See example 1).

Within the identified epitope patterns some amino acids are conservative, called anchor amino acids. The anchor amino acids recur in all or a majority of the reactive peptides.

Epitope Mapping Algorithm

When epitope patterns have been identified they are subsequently compared to the three-dimensional coordinates of the amino acid sequence of the polypeptide of interest, in order to identify combinations of residues on the polypeptide surface corresponding to the consensus sequence(s) or epitope pattern(s). In this way, amino acids residues, which are important for antibody binding, can be identified.

Once one or more epitope patterns have been identified, any polypeptide for which a three-dimensional structure is known may be analysed for epitopes matching the epitope patterns. Finding an epitope on a polypeptide is achieved by searching the surface of the polypeptide in the following way:

(1) For all amino acids in the polypeptide it is examined if (a) the amino acid type match the first amino acid of an epitope pattern and (b) the surface accessibility greater than or equal to a chosen threshold allowing the amino acid to be immunological interactive. Those amino acid satisfying 1 (a) and 1 (b) are selected.

(2) For all amino acids within a selected distance (e.g. 10 Angstroms) of the amino acids selected in step 1 it is examined if (a) the amino acid type matches the second amino acid of the pattern and (b) the surface accessibility greater than or equal to a chosen threshold allowing the amino acid to be immunological interactive. Those amino acid satisfying 2(a) and 2(b) are selected.

(3) For all amino acids within a selected distance (e.g., 10 Angstroms) of the amino acids selected in step 2 it is examined if (a) the amino acid type matches the third amino acid of the pattern and (b) the surface accessibility greater than or equal to a chosen threshold allowing the amino acid to be immunological interactive. Those amino acid satisfying 3(a) and 3(b) are selected.

This procedure (step 3) is repeated for all amino acids in the epitope pattern consensus sequence. The coordinates of its C-alpha atom define the spatial positioning of an amino acid. The surface solvent accessibility threshold is given in percent of an average for the particular residue type (see example 2).

If matching amino acids for all amino acids in the epitope pattern can be found in the structure of the polypeptide it is a very strong indication that an epitope has been found.

However, it is also checked that the size of the epitope is satisfactory, i.e., the distance between any two residues is below a given threshold, usually 25 Å.

The epitopes found may be ranked and weighted according to their total accessible surface area, in order to improve further the predictability of the tool.

Finally, when all possible epitopes have been mapped for the protein of interest, one can provide a score for each amino acid of the protein by adding up the number of times it appears in an epitope pattern. This score will be an indication of the likelyhood that modification (substitution, insertion, deletion, glycosylation or chemical conjugation) of that amino acid will, result in a variant with a lower antigenicity. All amino acids of the protein can then be ranked according to this score and those with highest scores can be selected for mutagenesis.

The epitope mapping tool can be adjusted, such that only a subset of the known reactive peptides are included as data set for building epitope patterns, and thus for conducting epitope mapping. For instance, one may choose only to include peptides reactive to IgE antibodies (rather than to IgG or other antibodies), or one may include only peptides reactive to human antibodies etc. One may choose to involve only peptides reactive against the target protein in order to get a more specific response; however, in general, peptides reactive to antibodies that in turn were raised against any protein are included.

If no three-dimensional structure coordinates are available for the protein of interest, one can map the epitope patterns directly to the primary sequence of the protein of interest.

From all the above information, it is obvious that the epitopes are conveniently determined using this epitope mapping tool.

Further, the in silico epitope mapping tool can be used to predict if mutating one amino acid residue will result in that the new variant overall will have fewer epitopes. Thus, some or all 19 possible substitutions can be tested in a given position, the epitope mapping procedure repeated for a model structure of each of these proposed variants, and the best variant(s) can be constructed by mutation and tested experimentally.

Identified Epitopes of Group 1 Mite Polypeptides

Using the epitope mapping tool the present inventors have surprisingly found that amino acids corresponding to positions A10, A12, E13, G29, G30, G32, A46, Y47, L55, D64, A66, S67, G73, T75, I80, Q84, N86, G87, S92, Y93, Y96, A98-Q101, R104-P106, Q109-I113, A132, I144-D146, D148, R151, I158-Q166, N179, A180, G182-D184, A205, I208 of SEQ ID NO: 1 or alternatively 10, 12, 13, 29, 30, 32, 46, 47, 55, 64, 66, 67, 73, 75, 80, 84, 86, 87, 92, 93, 96, 98-101, 104-106, 109-113, 132, 144-146, 148, 151, 158-166, 179-180, 182-184, 205, 208 of the mature Der p 1 polypeptide in particularly that of SEQ ID NO: 1, are comprised in the epitopes of native group 1 mite polypeptides. Group 1 mite polypeptides are as stated above highly homologeous and the corresponding positions in Group 1 mite polypeptides of various sources may easily be found by aligning such polypeptides with SEQ ID NO: 1.

Group 1 Mite Polypeptide Variants

Selection of Positions for Mutation

Once the epitopes of a polypeptide have been determined, variants of the polypeptide with modified antigenic properties can be made by mutating one or more of the amino acid residues comprised in the epitope. In this context mutation encompasses deletion and/or substitution of an amino acid residue and/or insertion of one or more amino acids before or after that residue.

When providing a polypeptide variant suitable as a vaccine agent, for treatment of allergies, it is particularly desirable to alter IgE epitopes to reduce the binding of IgE, while at the same time maintaining the ability of the variant to invoke an immune response in humans and animals, hence it is desirable that the polypeptides retain the three-dimensional conformation of the parent polypeptide. Further, it is particularly desirable that the variant is capable of stimulating T-cells sufficiently, preferable at the level of the parent polypeptide or better. Still further it is desirable that the variant is capable of invoking an IgG response in human and animals.

The epitope identified may be mutated by substituting at least one amino acid of the epitope. In a particular embodiment at least one anchor amino acid is mutated. The mutation will often be a substitution with an amino acid of different size, hydrophilicity, polarity and/or acidity, such as a small amino acid in exchange of a large amino acid, a hydrophilic amino acid in exchange of a hydrophobic amino acid, a polar amino acid in exchange of a non-polar amino acid and a basic in exchange of an acidic amino acid.

Other mutations may be the insertion or deletion of at least one amino acid of the epitope, particularly deleting an anchor amino acid. Furthermore, an epitope may be mutated by substituting some amino acids, and deleting and/or inserting others.

The mutation(s) performed may be performed by standard techniques well known to a person skilled in the art, such as site-directed mutagenesis (see, e.g., Sambrook et al. (1989), Molecular Cloning. A Laboratory Manual, Cold Spring Harbor, N.Y.).

The mutagenesis may be spiked mutagenesis which is a form of site-directed mutagenesis, in which the primers used have been synthesized using mixtures of oligonucleotides at one or more positions.

A general description of nucleotide substitution can be found in e.g., Ford et al., 1991, Protein Expression and Purification 2, pp. 95-107.

The polypeptide variant of the invention concerns variants of parent group 1 mite polypeptides comprising one or more mutations in the parent polypeptide in the positions or corresponding to the positions consisting of A10, A12, E13, G29, G30, G32, A46, Y47, S54, L55, D64, A66, S67, G73, T75, I80, Q84, N86, G87, S92, Y93, Y96, A98, R99, E100, Q101, R104, R105, P106, Q109, R110, F111, G112, I113, A132, I144, K145, D146, D148, R151, I158, I159, Q160, R161, D162, N163, G164, Y165, Q166, N179, A180, G182, V183, D184, A205, I208 of SEQ ID NO: 1 or 10, 12, 13, 29, 30, 32, 46, 47, 54, 55, 64, 66, 67, 73, 75, I80, 84, 86, 87, 92, Y93, 96, 98, 99, 100, 101, 104, 105, 106, 109, 110, 111, 112, 113, 132, 144, 145, 146, 148, 151, 158, 159, 160, 161, 162, 163, 164, 165, 166, 179, 180, 182, 183, 184, 205, 208 of the mature Der p 1 polypeptide.

In another particular embodiment of the above the variant polypeptide of the invention comprises one or more mutations in the parent polypeptide in the positions or corresponding to the positions consisting of A10, A12, G29, G30, G32, A46, Y47, S54, L55, D64, A66, G73, T75, I80, Q84, N86, G87, S92, Y93, Y96, A98, E100, Q101, R104, R105, P106, R110, F111, G112, I113, A132, I144, K145, D146, I158, I159, Q160, D162, N163, G164, Y165, Q166, N179, A180, G182, V183, D184, A205, I208 of SEQ ID NO: 1 or 10, 12, 29, 30, 32, 46, 47, 55, 64, 66, 73, 75, I80, 84, 86, 87, 92, Y93, 96, 98, 100, 101, 104, 105, 106, 110, 111, 112, 113, 132, 144, 145, 146, 158, 159, 160, 162, 163, 164, 165, 166, 179, 180, 182, 183, 184, 205, 208 of the mature Der p 1 polypeptide.

In still another particular embodiment the variant polypeptide of the invention comprises one or more mutations in the parent polypeptide in the positions or corresponding to the positions consisting of A10, A12, G29, G30, G32, A46, S54, L55, D64, A66, G73, T75, Q84, N86, G87, S92, Y96, E100, Q101, R104, R105, P106, R110, I113, A132, I144, K145, D146, D162, N163, G164, Y165, N179, A180, G182, V183, D184 and A205 of SEQ ID NO: 1 or 10, 12, 29, 30, 32, 46, 55, 64, 66, 73, 75, 84, 86, 87, 92, 96, 100, 101, 104, 105, 106, 110, 113, 132, 144, 145, 146, 162, 163, 164, 165, 179, 180, 182, 183, 184 and 205 of the mature Der p 1 polypeptide.

Particularly the variant has an altered antibody binding profile as compared the parent group 1 mite polypeptide, more particularly the variant has a reduced IgE-binding, more particularly combined with preserved immunogenicity for inducing protective responses (vide supra). Still more preferably, the variant has an altered immunogenic profile in exposed animals, including humans, as compared to the parent group 1 mite polypeptide.

Further the variant has in particular an altered IgE-antigenicity as compared to the parent group 1 mite polypeptide.

Still further the variant has in particular at least the same T-cell stimulatory effect compared to the parent group 1 mite polypeptide as measured by the procedure in example 6.

Still further the variant induces an altered immunogenic response in exposed animals, including humans, as compared to the parent group 1 mite polypeptide.

Still further the variant induces in particular an altered immunogenic response in humans, as compared to the parent group 1 mite polypeptide.

In still another particular embodiment the variant polypeptide of the invention comprises one or more mutations in the parent polypeptide in the positions or corresponding to the positions consisting of A10, A12, G30, G32, A46, Y47, S54, L55, D64, A66, S67, G87, S92, A98, R99, E100, Q101, R105, R110, F111, G112, I113, I144, K145, D146, D148, R151, I159, Q160, R161, D162, N163, G164, Y165, Q166, N179, A180, G182, V183, D184, A205, I208 of SEQ ID NO: 1 or 10, 12, 30, 32, 46, 47, 54, 55, 64, 66, 67, 87, 92, 98, 99, 100, 101, 105, 110, 111, 112, 113, 144, 145, 146, 148, 151, 159, 160, 161, 162, 163, 164, 165, 166, 179, 180, 182, 183, 184, 205, 208 of the mature Der p 1 polypeptide.

In still another particular embodiment of the above the variant polypeptide of the invention comprises one or more mutations in the parent polypeptide in the positions or corresponding to the positions consisting of A10, A12, G32, S54, L55, A66, S67, G87, A98, R99, F111, G112, I113, I144, D146, D148, I159, R161, G164, Q166, A180, D184, A205 and I208 of SEQ ID NO: 1 or 10, 12, 32, 54, 55, 66, 67, 87, 98, 99, 111, 112, 113, 144, 146, 148, 159, 161, 164, 166, 180, 184, 205 and 208 of the mature Der p 1 polypeptide.

Mutations Directly Providing for Reduced Antigenicity

When providing mutants having a reduced antigenicity it may be particularly interesting to substitute an amino acid in an epitope of a parent group 1 mite polypeptide with an amino acid having different properties. Hence, in a particular embodiment, the variant polypeptide of the invention comprises a mutation selected from the group consisting of
10 substituted by a residue selected from the group consisting of V, Y, Q, N, E and D;
12 substituted by a residue selected from the group consisting of V, Y, Q, N and F;
32 substituted by a residue selected from the group consisting of V, Y, E, D, N and Q;
54 substituted by a residue selected from the group consisting of N, A, T, V, and Q;
55 substituted by a residue selected from the group consisting of V, N, and Q;
66 substituted by a residue selected from the group consisting of V, H, Y, D, E, N, and Q;
67 substituted by a residue selected from the group consisting of V, H, Y, D, E, N and Q;
87 substituted by a residue selected from the group consisting of V, Y, D and E;
98 substituted by a residue selected from the group consisting of V, N, Q, D or E;
99 substituted by a residue selected from the group consisting of H, Y, V, N, Q, E and D;
111 substituted by a residue selected from the group consisting of V, H and W;
112 substituted by a residue selected from the group consisting of V, H, N, Q, E, D and Y;
113 substituted by a residue selected from the group consisting of V, H, N, Q, E, D and Y;
144 substituted by a residue selected from the group consisting of A, G, Y, N, Q or V;
146 substituted by a residue selected from the group consisting of Y, H, V, I, L, N and Q;
148 substituted by a residue selected from the group consisting of Y, V, I and L;
159 substituted by a residue selected from the group consisting of V, Y, A and G;
161 substituted by a residue selected from the group consisting of A, G, V and Y;
164 substituted by a residue selected from the group consisting of V, H and W;
166 substituted by a residue selected from the group consisting of S, W, Y and F;
180 substituted by a residue selected from the group consisting of V, N, Q and Y;
184 substituted by a residue selected from the group consisting of V, M and Y;
205 substituted by a residue selected from the group consisting of V, W and H;
208 substituted by a residue selected from the group consisting of A, G, V, W and H;
   said numbering being positions of or corresponding to positions of the mature Der p1 polypeptide.

In still another particular embodiment the variant polypeptide of the invention comprises a mutation selected from the group consisting of
A10 substituted by a residue selected from the group consisting of V, Y, Q, N, E and D;
A12 substituted by a residue selected from the group consisting of V, Y, Q, N and F;
G32 substituted by a residue selected from the group consisting of V, Y, E, D, N and Q;
S54 substituted by a residue selected from the group consisting of N, A, T, V, and Q;
L55 substituted by a residue selected from the group consisting of V, N and Q;
A66 substituted by a residue selected from the group consisting of V, H, Y, D, E, N and Q;
S67 substituted by a residue selected from the group consisting of V, H, Y, D, E, N and Q;
G87 substituted by a residue selected from the group consisting of V, Y, D and E;
A98 substituted by a residue selected from the group consisting of V, N, Q, D or E;
R99 substituted by a residue selected from the group consisting of H, Y, V, N, Q, E and D;
F111 substituted by a residue selected from the group consisting of V, H and W;
G112 substituted by a residue selected from the group consisting of V, H, N, Q, E, D and Y;
I113 substituted by a residue selected from the group consisting of V, H, N, Q, E, D and Y;
I144 substituted by a residue selected from the group consisting of A, G, Y, N, Q or V;
D146 substituted by a residue selected from the group consisting of Y, H, V, I, L, N and Q;
D148 substituted by a residue selected from the group consisting of Y, V, I and L;
I159 substituted by a residue selected from the group consisting of V, Y, A and G;
R161 substituted by a residue selected from the group consisting of A, G, V and Y;
G164 substituted by a residue selected from the group consisting of V, H and W;
Q166 substituted by a residue selected from the group consisting of S, W, Y and F;
A180 substituted by a residue selected from the group consisting of V, N, Q and Y;
D184 substituted by a residue selected from the group consisting of V, M and Y;
A205 substituted by a residue selected from the group consisting of V, W and H;
I208 substituted by a residue selected from the group consisting of A, G, V, W and H;
   said numbering being positions of or corresponding to positions of SEQ ID NO: 1.

In a further embodiment the parent group 1 mite polypeptide has in its mature form a sequence which displays at least 80% identity to SEQ ID NO: 1; in particular at least 90% identity; in particular at least 95% identity, more particularly 98% identity, more particularly 100% identity to SEQ ID NO: 1 or 100% identity to Der p 1.

In a further embodiment the variant group 1 mite polypeptide has in its mature form a sequence which displays at least 80% identity to SEQ ID NO: 1; in particular at least 90% identity; in particular at least 95% identity, more particularly 98% identity, more particularly 100% identity to SEQ ID NO: 1 or 100% identity to Der p 1.

In a further embodiment the parent group 1 mite polypeptide has in its mature form a sequence which displays at least 80% identity to SEQ ID NO: 2; in particular at least 90% identity; in particular at least 95% identity, more particularly 98% identity, more particularly 100% identity to SEQ ID NO: 2 or 100% identity to Eur m1.

In a further embodiment the variant group 1 mite polypeptide has in its mature form a sequence which displays at least 80% identity to SEQ ID NO: 2; in particular at least 90% identity; in particular at least 95% identity, more particularly 98% identity, more particularly 100% identity to SEQ ID NO: 2 or 100% identity to Eur m1.

In a further embodiment the parent group 1 mite polypeptide has in its mature form a sequence which displays at least 80% identity to SEQ ID NO: 3; in particular at least 90% identity; in particular at least 95% identity, more particularly 98% identity, more particularly 100% identity to SEQ ID NO: 3 or 100% identity to Der f1.

In a further embodiment the variant group 1 mite polypeptide has in its mature form a sequence which displays at least 80% identity to SEQ ID NO: 3; in particular at least 90% identity; in particular at least 95% identity, more particularly 98% identity, more particularly 100% identity to SEQ ID NO: 3 or 100% identity to Der f1.

In a further embodiment the parent group 1 mite polypeptide has in its mature form a sequence which displays at least 80% identity to Der m1; in particular at least 90% identity; in particular at least 95% identity, more particularly 98% identity; more particularly 100% identity to Der m1.

In a further embodiment the variant group 1 mite polypeptide has in its mature form a sequence which displays at least 80% identity to Der m1; in particular at least 90% identity; in particular at least 95% identity, more particularly 98% identity; more particularly 100% identity to Der m1.

In a further embodiment the parent group 1 mite polypeptide has in its mature form a sequence which displays at least 80% identity to SEQ ID NO: 5; in particular at least 90% identity; in particular at least 95% identity, more particularly 98% identity, more particularly 100% identity to SEQ ID NO: 5 or 100% identity to Blo t 1.

In a further embodiment the variant group 1 mite polypeptide has in its mature form a sequence which displays at least 80% identity to SEQ ID NO: 5; in particular at least 90% identity; in particular at least 95% identity, more particularly 98% identity, more particularly 100% identity to SEQ ID NO: 5 or 100% identity to Blo t 1.

The risk linked to protein engineering in order to eliminate epitopes that new epitopes are made, or existing epitopes are duplicated is reduced by testing the planned mutations at a given position in the 3-dimensional structure of the protein of interest against the found epitope patterns thereby identifying the mutations for each position that are feasible for obtaining the desired properties of the polypeptide.

In a particular embodiment of the invention, it will be an advantage, to establish a library of diversified mutants each having one or more changed amino acids introduced and selecting those variants, which show the best effect as vaccine agent while the fewest side effects. A diversified library can be established by a range of techniques known to the person skilled in the art (Reetz M T; Jaeger K E, in Biocatalysis—from Discovery to Application edited by Fessner W D, Vol. 200, pp. 31-57 (1999); Stemmer, Nature, vol. 370, p. 389-391, 1994; Zhao and Arnold, Proc. Natl. Acad. Sci., USA, vol. 94, pp. 7997-8000, 1997; or Yano et al., Proc. Natl. Acad. Sci., USA, vol. 95, pp 5511-5515, 1998). In a more preferable embodiment, substitutions are found by a method comprising the following steps: 1) a range of substitutions, additions, and/or deletions are listed encompassing several epitopes, 2) a library is designed which introduces a randomized subset of these changes in the amino acid sequence into the target gene, e.g., by spiked mutagenesis, 3) the library is expressed, and preferred variants are selected. In a most preferred embodiment, this method is supplemented with additonal rounds of screening and/or family shuffling of hits from the first round of screening (J. E. Ness, et al, Nature Biotechnology, Vol. 17, pp. 893-896, 1999).

Mutations Providing for Increased Glycosylation to Amino Acids in the Epitope Area

In another approach, the mutations are designed, such that recognition sites for post-translational modifications are introduced in the epitope areas, and the protein variant is expressed in a suitable host organism capable of the corresponding post-translational modification. These post-translational modifications may serve to shield the epitope and hence lower the immunogenicity of the protein variant relative to the protein backbone. Post-translational modifications include glycosylation, phosphorylation, N-terminal processing, acylation, ribosylation and sulfatation. A good example is N-glycosylation. N-glycosylation is found at sites of the sequence Asn-Xaa-Ser, Asn-Xaa-Thr, or Asn-Xaa-Cys, in which neither the Xaa residue nor the amino acid following the tri-peptide consensus sequence is a proline (T. E. Creighton, “Proteins—Structures and Molecular Properties, 2nd edition, W.H. Freeman and Co., New York, 1993, pp. 91-93). It is thus desirable to introduce such recognition sites in the sequence of the backbone protein. The specific nature of the glycosyl chain of the glycosylated protein variant may be linear or branched depending on the protein and the host cells. Another example is phosphorylation: The protein sequence can be modified so as to introduce serine phophorylation sites with the recognition sequence arg-arg-(xaa)ₙ-ser (where n=0, 1, or 2), which can be phosphorylated by the cAMP-dependent kinase or tyrosine phosphorylation sites with the recognition sequence -lys/arg-(xaa)₃-asp/glu-(xaa)₃-tyr, which can usually be phophorylated by tyrosine-specific kinases (T. E. Creighton, “Proteins—Structures and molecular properties”, 2nd ed., Freeman, N.Y., 1993).

Mutations Providing for Covalent Conjugation of Polymers to Amino Acids in the Epitope Area

Another way of making mutations that will change the antigenic properties of a polypeptide is to react or conjugate polymers to amino acids in or near the epitope, thus blocking or shielding the access to the anchor amino acids and thus the binding of antibodies and/or recptors to those amino acids. If no amino acid suitable for conjugation with a polymer exists in the parent polypeptide a suitable mutation is the insertion of one or more amino acids being attachment sites and/or groups and/or amino acids for polymer conjugation.

Which amino acids to substitute and/or insert depends in principle on the coupling chemistry to be applied. The chemistry for preparation of covalent bioconjugates can be found in “Bioconjugate Techniques”, Hermanson, G. T. (1996), Academic Press Inc., which is hereby incorporated as reference.

It a particular embodiment activated polymers are conjugated to amino acids in or near the epitope area.

It is preferred to make conservative substitutions in the polypeptide when the polypeptide has to be conjugated, as conservative substitutions secure that the impact of the substitution on the polypeptide structure is limited.

In the case of providing additional amino groups, this may be done by substitution of Arginine to Lysine, both residues being positively charged, but only the Lysine having a free amino group suitable as an attachment groups.

In the case of providing additional carboxylic acid groups, the conservative substitution may for instance be an Asparagine to Aspartic acid or Glutamine to Glutamic acid substitution. These residues resemble each other in size and shape, except from the carboxylic groups being present on the acidic residues.

In the case of providing SH-groups the conservative substitution may be done by substitution of Threonine or Serine to Cysteine.

Verification of Variants Having Altered Antigenic Properties

The mutation of amino acids, comprised in an epitope, will cause the antigenic properties of the polypeptide to change, as predicted by the in silico determination of the epitopes. However, the quantitative effect of the mutation on the antigenicity, i.e., the antibodybinding, and the immunogenicity of the variant, is suitably determined using various in vivo or in vitro model systems. For that use, the polypeptide variant of interest can be expressed in larger scale and purified by conventional techniques. Then the functionality and specific activity may be tested by cysteine protease activity assays, in order to assure that the variant has retained three-dimensional structure.

In vitro systems include assays measuring binding to IgE in serum from dust mite allergic patients or exposed animals, cytokine expression profiles or proliferation responses of primary T-cells from dust mite allergic patients or T cell clones or T cell line generated from dust mite allergic patients (Current protocols in Immunology, chapter 7 and 9), or exposed animals, and histamine release from basophils from dust mite allergic patients.

The IgE antibody binding can be examined in detail using, e.g., direct or competitive ELISA (C-ELISA), histamine release assays on basophil cells from allergic patients, or IgE-stripped basophils from whole blood incubated with IgE-containing serum from allergic patients, or by other or other solid phase immunoassays or cellular assays (see Example 9). The use of stripped basophils from whole blood is described in: Knol E F, Kuijpers T W, Mul F P, Roos D. Stimulation of human basophils results in homotypic aggregation. A response independent of degranulation. J. Immunol. 1993 Nov. 1; 151(9): 4926-33; and in: Budde I K, Aalbers M, Aalberse R C, van der Zee J S, Knol E F. Reactivity to IgE-dependent histamine-releasing factor is due to monomeric IgE. Allergy. 2000 July; 55(7): 653-7.

In a particular embodiment the ability of the polypeptide variant to bind IgE is reduced at least 3 times as compared to the binding ability of the orginal or parent group 1 mite polypeptide, preferably 5 times reduced, more preferably 10 times reduced, or more preferably 50 times reduced.

In a further particular embodiment the ability of the polypeptide variant to induce histamine release in basophil cells from subjects allergic to dust mites is reduced least 3 times, as compared to that of the parent group 1 mite polypeptide, preferably 10 times reduced, more preferably 50 times.

In a further embodiment, the ability of the polypeptide variant to invoke a recall T-cell response in lymphocytes from animals, including humans, previously exposed to the orginal or parent group 1 mite allergen is measured, preferably the strength of the response is comparable to or higher than that to the parent group 1 mite allergen.

In a particular embodiment the in vivo verification comprises skin prick testing (SPT), in which a dust mite allergic subject/indvidual is exposed to intradermal or subcutaneous injection of group 1 mite polypeptides and the IgE reactivity, measured as the diameter of the wheal and flare reaction, in response to a polypeptide variant of the invention is compared to that to the parent group 1 mite polypeptide (Kronquist et al., Clin. Exp. Allergy, 2000, vol. 30, pp. 670-676).

Animal Models of Allergy and SIT—Experimental Immunization of Animals with Each of the Compositions

The in vivo immunogenic properties of the polypeptide variant of the invention may suitably be measured in an animal test, wherein test animals are exposed to a vaccination allergen polypeptide and the responses are measured and compared to those of the target allergen or other appropriate references. The immune response measurements may include comparing reactivity of serum IgG, IgE or T-cells from a test animal with target polypeptide and the polypeptide variant. Animal immunization can be conducted in at least two distinct manners: on naïve animals and on pre-sensitized animals (to better simulate the vaccine situation). In the context of this invention affinity of immunoglobulins towards the target antigen is tested.

In a particular embodiment the affinity of animal IgG and/or IgG1 and/or IgG4 following administration of the variant molecule is tested.

In the method according to the invention the test animals can either be naive animals or pre-sensitized animals.

A number of model systems are based on the use of naïve animals.

In a particular embodiment the in vivo verification comprises exposing a mouse to a parent target allergen by the intranasal route. Useful in vivo animal models include the mouse intranasal test (MINT) model (Robinson et al., Fund. Appl. Toxicol., Vol. 34, pp. 15-24, 1996).

In a further particular embodiment the in vivo verification comprises exposing a test animal to a polypeptide variant by the intratracheal route. Useful in vivo animal models include the guinea pig intratracheal (GPIT) model (Ritz, et al. Fund. Appl. Toxicol., Vol. 21, pp. 31-37, 1993) and the rat intratracheal (rat-IT) model (WO 96/17929, Novo Nordisk).

In a further particular embodiment, the in vivo verification comprises exposing a test animal subcutaneously to the target allergen and the vaccination allergen variant. A suitable model is the mouse subcutaneous (mouse-SC) model (WO 98/30682, Novo Nordisk).

In a further particular embodiment, the method comprises exposing the test animal intraperitoneally. ALK-Abelló disclose (WO02/40676) a method to assess the ability of allergen variants (of the birch pollen allergen bet v 1) to induce IgG antibodies upon immunization of mice: BALB/C mice were immunized intraperitoneally with the relevant allergy variant or controls, four times at dose intervals of 14 days. The proteins were conjugated to 1.25 mg/mL alhydogel (AlOH gel, 1.3%, pH8-8.4, Superfos Biosector). The mice were immunized with either 1 or 10 ug protein/dose. Blood samples were drawn at day 0, 14, 21, 35, 49, and 63 and analysed by direct ELISA using rBet v 1 coated microtiterplates and biotinylated rabbit anti mouse IgG antibodies as detecting antibodies.

In yet a further embodiment, the method comprise using transgenic mice capable of facilitating production of donor-specific immunity as test animals. Such mice are disclosed by Genencor International (WO 01/15521).

Also, a number of studies have assessed the effect of allergy vaccination compositions in animal models, in which the animals were sensitized to the relevant allergen prior to exposure to the vaccination composition:

Mice: Li et al. (J. Allergy Clin. Immunol., Vol. 112, pp. 159-167, 2003) disclose a mice-based system to assess efficacy of allergy vaccines. The mice are sensitized intra-gastrically with a food allergen, and the treatment is introduced as an intra-rectum injection. In a separate allegy vaccination system, Hardy et al. (AM J. Respir. Crit Care Med, vol 167, pp. 1393-1399, 2003) show that mice can be sensitized by intraperitonal injection, and that allergy vaccine compounds can be administered intrtracheally with the animals anaestethized. Sudowe et al., (Gene Therapy, Vol. 9, pp. 147-156, 2002) show that intraperitoneal injection in mice could be made to produce either TH1 or TH2 responses.

Rats: Wheeler et al., (Int. Arch. Allergy Immunol, Vol. 126, pp. 135-139, 2001) disclose a rat allergy model in which rats are injected subcutaneously along with adjuvant. These ‘allergic’ rats can then be made to conduct an allergy-vaccine like response, when subjected to subsequent injections with trial vaccine compositions.

Guinea Pigs: Nakamoto et al., (Clin Exp. Allergy, Vol. 27, pp. 1103-1108, 1997) demonstrate the use of guinea pigs as model system for SIT. Guinea pigs were injected intraperitoneally and boosted twice, and then they were exposed to the vaccine compound to register decreases in allergenicity by measuring antibody titers as a function of the compound, formulation, or mode of application.

Preparation of Nucleotide Constructs, Vectors, Host Cells, Protein Variants and Polymers for Conjugation

In accordance with the present invention there may be employed conventional molecular biology, microbiology, and recombinant DNA techniques well known to a person skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II/D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

The method may in a particular embodiment be carried out to express group 1 dust mite proteins as inclusion bodies in E. coli or in soluble form in methylotrophic yeasts such as Pichia pastoris, as described in WO 01/29078 (HESKA) describing recombinant expression of group 1 mite proteins including nucleotide sequences modified to enable expression of the polypeptides in microorganisms.

A preferred method is to express the group 1 dust mite proteins in S. cerevisiae cells, as described by Chua et al. (J. Allergy Clin Immunol., 1992, Vol. 89, pp. 95-102).

Another preferred method is to express group 1 dust mite proteins in insect cells such as Drosophila (Jacquet et al, Clin Exp. Allergy, 2000, Vol. 30, pp. 677-84) or Spodoptera frugiperda Sf9 cells infected with a bacullovirus system (Shoji, et al., Biosci. Biotech. Biochem., 1996, Vol. 60, pp. 621-25).

Nucleotide Sequences

The present invention also encompasses a nucleotide sequence encoding a polypeptide variant of the invention. As described, a description of standard mutation of nucleotide sequences to encode polypeptide variants by nucleotide substitution can be found in e.g., Ford et al., 1991, Protein Expression and Purification 2, p. 95-107. Other standard methods, such as site-directed mutagenesis is described in e.g., Sambrook et al. (1989), Molecular Cloning. A Laboratory Manual, Cold Spring Harbor, N.Y.

A “nucleotide sequence” is a single- or double-stranded polymer of deoxyribonucleotide or ribonucleotide bases read from the 5′ to the 3′ end. Nucleotide sequences include RNA and DNA, and may be isolated from natural sources, synthesized in vitro, or prepared from a combination of natural and synthetic molecules.

The techniques used to isolate or clone a nucleotide sequence alias a nucleotide sequence encoding a polypeptide are known in the art and include isolation from genomic DNA, preparation from cDNA, or a combination thereof. The cloning of the nucleotide sequences of the present invention from such genomic DNA can be effected, e.g., by using the well known polymerase chain reaction (PCR) or antibody screening of expression libraries to detect cloned DNA fragments with shared structural features. See, e.g. Innis et al., 1990, A Guide to Methods and Application, Academic Press, New York. Other nucleotide amplification procedures such as ligase chain reaction (LCR), ligated activated transcription (LAT) and nuceic acid sequence-based amplification (NASBA) may be used. The nucleotide sequence may be cloned from a strain producing the polypeptide, or from another related organism and thus, for example, may be an allelic or species variant of the polypeptide encoding region of the nucleotide sequence.

The term “isolated” nucleotide sequence as used herein refers to a nucleotide sequence which is essentially free of other nucleotide sequences, e.g., at least about 20% pure, preferably at least about 40% pure, more preferably about 60% pure, even more preferably about 80% pure, most preferably about 90% pure, and even most preferably about 95% pure, as determined by agarose gel electorphoresis. For example, an isolated nucleotide sequence can be obtained by standard cloning procedures used in genetic engineering to relocate the nucleotide sequence from its natural location to a different site where it will be reproduced. The cloning procedures may involve excision and isolation of a desired nucleotide fragment comprising the nucleotide sequence encoding the polypeptide, insertion of the fragment into a vector molecule, and incorporation of the recombinant vector into a host cell where multiple copies or clones of the nucleotide sequence will be replicated. The nucleotide sequence may be of genomic, cDNA, RNA, semisynthetic, synthetic origin, or any combinations thereof. Such isolated molecules are those that are separated from their natural environment and include cDNA and genomic clones. Isolated DNA molecules of the present invention are free of other genes with which they are ordinarily associated, and may include naturally occurring 5′ and 3′ untranslated regions such as promoters and terminators. The identification of associated regions will be evident to one of ordinary skill in the art (see for example, Dynan and Tijan, Nature 316: 774-78, 1985).

Nucleotide Construct

As used herein the term “nucleotide construct” is intended to indicate any nucleotide molecule of cDNA, genomic DNA, synthetic DNA or RNA origin. The term “construct” is intended to indicate a nucleotide segment which may be single- or double-stranded, and which may be based on a complete or partial naturally occurring nucleotide sequence encoding a polypeptide of interest. The construct may optionally contain other nucleotide segments.

The DNA of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleotide construct may also be prepared synthetically by established standard methods, e.g., the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g., in an automatic DNA synthesizer, purified, annealed, ligated and cloned in suitable vectors.

Furthermore, the nucleotide construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleotide construct, in accordance with standard techniques.

The nucleotide construct may also be prepared by polymerase chain reaction using specific primers, for instance as described in U.S. Pat. No. 4,683,202 or Saiki et al., Science 239 (1988), 487-491.

The term nucleotide construct may be synonymous with the term expression cassette when the nucleotide construct contains all the control sequences required for expression of a coding sequence of the present invention.

The term “coding sequence” as defined herein is a sequence which is transcribed into mRNA and translated into a polypeptide of the present invention when placed under the control of the above mentioned control sequences. The boundaries of the coding sequence are generally determined by a translation start codon ATG at the 5′-terminus and a translation stop codon at the 3′-terminus. A coding sequence can include, but is not limited to, DNA, cDNA, and recombinant nucleotide sequences.

The term “control sequences” is defined herein to include all components which are necessary or advantageous for expression of the coding sequence of the nucleotide sequence. Each control sequence may be native or foreign to the nucleotide sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

The nucleotide constructs of the present invention may also comprise one or more nucleotide sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleotides encoding one or more of these factors are not necessarily in tandem with the nucleotide sequence encoding the polypeptide.

Propeptides

The control sequence may also be a propeptide coding region, which codes for an amino acid sequence positioned at the amino terminus of a polypeptide. The resultant polypeptide is known as a proenzyme or propolypeptide (or a zymogen in some cases).

A propolypeptide is generally inactive and can be converted to mature active polypeptide by catalytic or autocatalytic cleavage of the propeptide from the propolypeptide. In the present invention protease of the family of subtilisin-like serine proteases have proven useful for activation of propeptides. Subtilisin could be added to the crude cell supernatant, to filtron concentrated supernatant, or to material that had been purified by column chromatography. The subtilisin could be removed by an extra chromatography step or inactivated with barley chymotrypsin inhibitor (Ci-2A). The time of incubation could range from 1 to 21 or 24 hours. The pro-der p 1 or corresponding propeptide of the variants were cleaved at the native processing site (as verified by Edman degradation and N-terminal sequencing) to give the N-terminal sequence TNACSIN.

The preferred Subtilisins are Savinase™ (Subtilisin from Bacillus clausii. Novozymes commercial product.), BPN′ (Subtilisin Novo from Bacillus amyloliquefaciens, SwissProt:SUBT_BACAM see Siezen et al., Protein Engng. 4 (1991) 719-737), PD498 (Subtilisin from a Bacillus sp., GeneSeqP:AAW24071; WO9324623A1) and B34 (Subtilisin from Bacillus alcalophilus, patent WO 0158275). The most preferred is BPN′ (BASBPN) (Siezen et al., Protein Engng. 4 (1991) 719-737)) dosed to a final concentration ranging from 0.25 to 165 microg/ml, preferably 16.5 to 165 microgram/ml.

The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

Activators

An activator is a protein which activates transcription of a nucleotide sequence encoding a polypeptide (Kudla et al., 1990, EMBO Journal 9: 1355-1364; Jarai and Buxton, 1994, Current Genetics 26: 2238-244; Verdier, 1990, Yeast 6: 271-297). The nucleotide sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra and MacKenzie et al., 1993, Journal of General Microbiology 139: 2295-2307.

Chaperones

A chaperone is a protein which assists another polypeptide in folding properly (Hartl et al., 1994, TIBS 19: 20-25; Bergeron et al., 1994, TIBS 19: 124-128; Demolder et al., 1994, Journal of Biotechnology 32: 179-189; Craig, 1993, Science 260: 1902-1903; Gething and Sambrook, 1992, Nature 355: 33-45; Puig and Gilbert, 1994, Journal of Biological Chemistry 269: 7764-7771; Wang and Tsou, 1993, The FASEB Journal 7: 1515-11157; Robinson et al., 1994, Bio/Technology 1: 381-384). The nucleotide sequence encoding a chaperone may be obtained from the genes encoding Bacillus subtilis GroE proteins, Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, 1992, supra, and Hartl et al., 1994, supra.

Processing Protease

A processing protease is a protease that cleaves a propeptide to generate a mature biochemically active polypeptide (Enderlin and Ogrydziak, 1994, Yeast 10: 67-79; Fuller et al., 1989, Proceedings of the National Academy of Sciences USA 86: 1434-1438; Julius et al., 1984, Cell 37: 1075-1089; Julius et al., 1983, Cell 32: 839-852). The nucleotide sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xpr6).

Promoters

The control sequence may be an appropriate promoter sequence, a nucleotide sequence which is recognized by a host cell for expression of the nucleotide sequence. The promoter sequence contains transcription and translation control sequences which mediate the expression of the polypeptide. The promoter may be any nucleotide sequence which shows transcriptional activity in the host cell of choice and may be obtained from genes encoding extracellular or intracellular polypeptides either homologous or heterologous to the host cell.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

Examples of suitable promoters for directing the transcription of the nucleotide constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus subtilis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25), or the Bacillus pumilus xylosidase gene, or by the phage Lambda PR or PL promoters or the E. coli lac, trp or tac promoters. Further promoters are described in “Useful proteins from recombinant bacteria” in Scientific American, 1980, 242: 74-94; and in Sambrook et al., 1989, supra.

Examples of suitable promoters for directing the transcription of the nucleotide constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (-amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8: 423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for directing the transcription of the DNA encoding the polypeptide of the invention in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Terminators

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease for fungal hosts) the TPI1 (Alber and Kawasaki, op. cit.) or ADH3 (McKnight et al., op. cit.) terminators.

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, supra.

Polyadenylation Signals

The control sequence may also be a polyadenylation sequence, a sequence which is operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Useful polyadenylation sequences for yeast host cells are described by Guo and Sherman, 1995, Molecular Cellular Biology 15: 5983-5990.

Polyadenylation sequences are well known in the art for mammalian host cells such as SV40 or the adenovirus 5 Elb region.

Signal Sequences

The control sequence may also be a signal peptide coding region, which codes for an amino acid sequence linked to the amino terminus of the polypeptide which can direct the expressed polypeptide into the cell's secretory pathway of the host cell. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

A “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway.

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57: 109-137.

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase or lipase gene, or the Rhizomucor miehei lipase or protease gene, Aspergillus sp. amylase or glucoamylase, a gene encoding a Rhizomucor miehei lipase or protease. The signal peptide is preferably derived from a gene encoding A. oryzae TAKA amylase, A. niger neutral (alpha-amylase, A. niger acid-stable amylase, or A. niger glucoamylase.

Useful signal peptides for yeast host cells are obtained from the genes for Saccharomyces cerevisiae a-factor and Saccharomyces cerevisiae invertase. Other useful signal peptide coding regions are described by Romanos et al., 1992, supra.

For secretion from yeast cells, the secretory signal sequence may encode any signal peptide which ensures efficient direction of the expressed polypeptide into the secretory pathway of the cell. The signal peptide may be a naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and uptream of the DNA sequence encoding the polypeptide. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g., U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

Other Regulator Sequences

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GALL system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be placed in tandem with the regulatory sequence.

Recombinant Expression Vector Comprising Nucleotide Construct

The present invention also relates to a recombinant expression vector comprising a nucleotide sequence of the present invention, a promoter, and transcriptional and translational stop signals. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleotide construct comprising the sequence into an appropriate vector for expression. In creating the expression vector, the coding sequence is located in the vector so that the coding sequence is operably linked with the appropriate control sequences for expression, and possibly secretion.

“Operably linked”, when referring to DNA segments, indicates that the segments are arranged so that they function in concert for their intended purposes, e.g., transcription initiates in the promoter and proceeds through the coding segment to the terminator.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

The recombinant expression vector may be any vector (e.g., a plasmid or virus), which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol, tetracycline, neomycin, hygromycin or methotrexate resistance. A frequently used mammalian marker is the dihydrofolate reductase gene (DHFR). Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination. These nucleotide sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMβ1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75: 1433).

More than one copy of a nucleotide sequence encoding a polypeptide of the present invention may be inserted into the host cell to amplify expression of the nucleotide sequence. Stable amplification of the nucleotide sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al., 1989, supra).

Host Cell Comprising Nucleotide Constructs

The present invention also relates to recombinant host cells, comprising a nucleotide sequence or nucleotide construct or recombinant expression vector of the invention, which are advantageously used in the recombinant production of the polypeptide variants of the invention. The term “host cell” encompasses a parent host cell and any progeny thereof, which is not identical to the parent host cell due to mutations that occur during replication.

The cell is preferably transformed with a vector comprising a nucleotide sequence of the invention followed by integration of the vector into the host chromosome. “Transformation” means introducing a vector comprising a nucleotide sequence of the present invention into a host cell so that the vector is maintained as a chromosomal integrant or as a self-replicating extra-chromosomal vector. Integration is generally considered to be an advantage as the nucleotide sequence is more likely to be stably maintained in the cell. Integration of the vector into the host chromosome may occur by homologous or non-homologous recombination as described above.

The choice of a host cell will to a large extent depend upon the gene encoding the polypeptide and its source. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. The transformation of a bacterial host cell may, for instance, be effected by protoplast transformation (see, e.g., Chang and Cohen, 1979, Molecular General Genetics 168: 111-115), by using competent cells (see, e.g., Young and Spizizin, 1961, Journal of Bacteriology 81: 823-829, or Dubnar and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5771-5278).

The host cell may be a eukaryote, such as a mammalian cell, an insect cell, a plant cell or a fungal cell.

Useful mammalian cells include Chinese hamster ovary (CHO) cells, HeLa cells, baby hamster kidney (BHK) cells, COS cells, or any number of other immortalized cell lines available, e.g., from the American Type Culture Collection.

Examples of suitable mammalian cell lines are the COS (ATCC CRL 1650 and 1651), BHK (ATCC CRL 1632, 10314 and 1573, ATCC CCL 10), CHL (ATCC CCL39) or CHO (ATCC CCL 61) cell lines. Methods of transfecting mammalian cells and expressing DNA sequences introduced in the cells are described in e.g., Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

The yeast host cell may be selected from a cell of a species of Candida, Kluyveromyces, Saccharomyces, Schizosaccharomyces, Candida, Pichia, Hansenula, or Yarrowia. In a preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis, Kluyveromyces fragilis, Hansenula polymorpha, Pichia pastoris, Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. No. 4,882,279 and U.S. Pat. No. 4,879,231).

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligatelyu aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger, Aspergillus nidulans or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another preferred embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell or an Acremonium chrysogenum cell. In another most preferred embodiment, the Trichoderma cell is a Trichoderma harzianum, Trichoderma koningii, Trichoderma longibrachiatum, Trichoderma reesei or Trichoderma viride cell. The use of Aspergillus spp. for the expression of proteins is described in, e.g., EP 272 277, EP 230 023.

The nucleotide sequences, DNA, of the invention may be modified such as to optimize the codon usage for a preferred particular host organism in which it will be expressed. Examples of this are published for yeast (Woo J H, et al, Protein Expression and Purification, Vol. 25 (2), pp. 270-282, 2002), fungi (Te'o et al, FEMS Microbiology Letters, Vol. 190 (1) pp. 13-19 (2000)), and other microorganisms, as well as for Der p 1 expressed in mammalian cells (Massaer M, et al, International Archives of Allergy and Immunology, Vol. 125 (1), pp. 32-43, 2001).

In a particular embodiment the host cell is an insect cell and/or insect cell line. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

Plants

The present invention also relates to a transgenic plant, plant part, or plant cell which has been transformed with a nucleic acid sequence encoding a polypeptide (i.e. variant) of the present invention so as to express and produce the polypeptide in recoverable quantities. The polypeptide may be recovered from the plant or plant part. Alternatively, the plant or plant part containing the recombinant polypeptide may be used as such for vaccine purposes.

In a particular embodiment, the polypeptide is targeted to the endosperm storage vacuoles in seeds. This can be obtained by synthesizing it as a precursor with a suitable signal peptide, see Horvath et al in PNAS, Feb. 15, 2000, Vol. 97, no. 4, pp. 1914-1919.

The transgenic plant can be dicotyledonous (a dicot) or monocotyledonous (a monocot) or engineered variants thereof. Examples of monocot plants are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Low-phytate plants as described e.g. in U.S. Pat. No. 5,689,054 and U.S. Pat. No. 6,111,168 are examples of engineered plants.

Examples of plant parts are stem, callus, leaves, root, fruits, seeds, and tubers. Also specific plant tissues, such as chloroplast, apoplast, mitochondria, vacuole, peroxisomes, and cytoplasm are considered to be a plant part. Furthermore, any plant cell, whatever the tissue origin, is considered to be a plant part.

Also included within the scope of the present invention are the progeny of such plants, plant parts and plant cells.

The transgenic plant or plant cell expressing a polypeptide of the present invention may be constructed in accordance with methods known in the art. Briefly, the plant or plant cell is constructed by incorporating one or more expression constructs encoding a polypeptide of the present invention into the plant host genome and propagating the resulting modified plant or plant cell into a transgenic plant or plant cell.

Conveniently, the expression construct is a nucleic acid construct which comprises a nucleic acid sequence encoding a polypeptide of the present invention operably linked with appropriate regulatory sequences required for expression of the nucleic acid sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences are determined, for example, on the basis of when, where, and how the polypeptide is desired to be expressed. For instance, the expression of the gene encoding a polypeptide of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV promoter may be used (Franck et al., 1980, Cell 21: 285-294). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588).

A promoter enhancer element may also be used to achieve higher expression of the variant of the present invention in the plant. For instance, the promoter enhancer element may be an intron which is placed between the promoter and the nucleotide sequence encoding a polypeptide of the present invention. For instance, Xu et al., 1993, supra disclose the use of the first intron of the rice actin 1 gene to enhance expression.

Still further, the codon usage may be optimized for the plant species in question to improve expression (see Horvath et al referred to above).

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38). However it can also be used for transforming monocots, although other transformation methods are generally preferred for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated therein the expression construct are selected and regenerated into whole plants according to methods well-known in the art.

The present invention also relates to methods for producing a polypeptide of the present invention comprising (a) cultivating a transgenic plant or a plant cell comprising a nucleic acid sequence encoding a variant of the present invention under conditions conducive for production of the variant; and (b) recovering the variant.

Methods of Preparing Group 1 Mite Polypeptide Variants

The polypetide variants of the invention may be prepared by (a) transforming a suitable host cell with a nucleotide construct of the invention, (b) cultivating the recombinant host cell of the invention comprising a nucleotide construct of the invention under conditions conducive for production of the variant of the invention and (c) recovering the variant. The method may in a particular embodiment be carried out as described in WO 01/29078 (HESKA) describing recombinant expression of group 1 mite proteins including nucleotide sequences modified to enable expression of the polypeptides in microorganisms.

Transformation

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. A suitable method of transforming Fusarium species is described by Malardier et al., 1989, Gene 78: 147-156 or in copending U.S. Ser. No. 08/269,449. Examples of other fungal cells are cells of filamentous fungi, e.g., Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78: 147-156.

Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920. Mammalian cells may be transformed by direct uptake using the calcium phosphate precipitation method of Graham and Van der Eb (1978, Virology 52: 546).

Transformation of insect cells and production of heterologous polypeptides therein may be performed as described in U.S. Pat. Nos. 4,745,051; 4,775,624; 4,879,236; 5,155,037; and 5,162,222; and EP 397,485) all of which are incorporated herein by reference.

Cultivation

The transformed or transfected host cells described above are cultured in a suitable nutrient medium under conditions permitting the production of the desired molecules, after which these are recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g., in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

Recovery

In a particular embodiment the polypeptide variant of the invention is in an isolated and purified form. Thus the polypeptide variant of the invention is provided in a preparation which more than 20% w/w pure, particularly more than 50% w/w pure, more particularly more than 75% w/w pure, more particularly more than 90% w/w pure and even more particularly more than 95% w/w pure. The purity in this context is to be understood as the amount of polypeptide variant of the invention present in the preparation of the total polypeptide material in the preparation.

When applied to a polypeptide, the term “isolated” indicates that the polypeptide is found in a condition other than its native environment, such as apart from blood and animal tissue. In a preferred form, the isolated polypeptide is substantially free of other proteins, particularly other proteins of animal origin. It is preferred to provide the polypeptides in a highly purified form, i.e., greater than 95% pure, more preferably greater than 99% pure.

If the molecules are secreted into the nutrient medium, they can be recovered directly from the medium. If they are not secreted, they can be recovered from cell lysates. The molecules are recovered from the culture medium by conventional procedures including separating the host cells from the medium by centrifugation or filtration, precipitating the proteinaceous components of the supernatant or filtrate by means of a salt, e.g., ammonium sulphate. The proteins may be matured in vitro e.g., by acidification to induce autocatalytic processing (Jacquet et al., Clin Exp Allergy, 2002, vol. 32 pp 1048-53), and they may be purified by a variety of chromatographic procedures, e.g., ion exchange chromatography, gelfiltration chromatography, affinity chromatography, or the like, dependent on the type of molecule in question (see, e.g., Protein Purification, J-C Janson and Lars Ryden, editors, VCH Publishers, New York, 1989).

Activation of Polymers

In case the variant of the invention is to be conjugated to one or more polymers and if the polymeric molecules to be conjugated with the polypeptide in question are not active it must be activated by the use of a suitable technique. It is also contemplated according to the invention to couple the polymeric molecules to the polypeptide through a linker. Suitable linkers are well-known to the skilled person.

Methods and chemistry for activation of polymeric molecules as well as for conjugation of polypeptides are intensively described in the literature. Commonly used methods for activation of insoluble polymers include activation of functional groups with cyanogen bromide, periodate, glutaraldehyde, biepoxides, epichlorohydrin, divinylsulfone, carbodiimide, sulfonyl halides, trichlorotriazine etc. (see R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). Some of the methods concern activation of insoluble polymers but are also applicable to activation of soluble polymers e.g., periodate, trichlorotriazine, sulfonylhalides, divinylsulfone, carbodiimide etc. The functional groups being amino, hydroxyl, thiol, carboxyl, aldehyde or sulfydryl on the polymer and the chosen attachment group on the protein must be considered in choosing the activation and conjugation chemistry which normally consist of i) activation of polymer, ii) conjugation, and iii) blocking of residual active groups.

In the following a number of suitable polymer activation methods will be described shortly. However, it is to be understood that also other methods may be used.

Coupling polymeric molecules to the free acid groups of polypeptides may be performed with the aid of diimide and for example amino-PEG or hydrazino-PEG (Pollak et al., (1976), J. Amr. Chem. Soc., 98, 289□291) or diazoacetate/amide (Wong et al., (1992), “Chemistry of Protein Conjugation and Crosslinking”, CRC Press).

Coupling polymeric molecules to hydroxy groups are generally very difficult as it must be performed in water. Usually hydrolysis predominates over reaction with hydroxyl groups.

Coupling polymeric molecules to free sulfhydryl groups can be reached with special groups like maleimido or the ortho-pyridyl disulfide. Also vinylsulfone (U.S. Pat. No. 5,414,135, (1995), Snow et al.) has a preference for sulfhydryl groups but is not as selective as the other mentioned.

Accessible Arginine residues in the polypeptide chain may be targeted by groups comprising two vicinal carbonyl groups.

Techniques involving coupling electrophilically activated PEGs to the amino groups of Lysines may also be useful. Many of the usual leaving groups for alcohols give rise to an amine linkage. For instance, alkyl sulfonates, such as tresylates (Nilsson et al., (1984), Methods in Enzymology vol. 104, Jacoby, W. B., Ed., Academic Press: Orlando, p. 56-66; Nilsson et al., (1987), Methods in Enzymology vol. 135; Mosbach, K., Ed.; Academic Press: Orlando, pp. 65-79; Scouten et al., (1987), Methods in Enzymology vol. 135, Mosbach, K., Ed., Academic Press: Orlando, 1987; pp 79-84; Crossland et al., (1971), J. Amr. Chem. Soc. 1971, 93, pp. 4217-4219), mesylates (Harris, (1985), supra; Harris et al., (1984), J. Polym. Sci. Polym. Chem. Ed. 22, pp. 341-352), aryl sulfonates like tosylates, and para-nitrobenzene sulfonates can be used.

Organic sulfonyl chlorides, e.g., Tresyl chloride, effectively converts hydroxy groups in a number of polymers, e.g., PEG, into good leaving groups (sulfonates) that, when reacted with nucleophiles like amino groups in polypeptides allow stable linkages to be formed between polymer and polypeptide. In addition to high conjugation yields, the reaction conditions are in general mild (neutral or slightly alkaline pH, to avoid denaturation and little or no disruption of activity), and satisfy the non-destructive requirements to the polypeptide.

Tosylate is more reactive than the mesylate but also more unstable decomposing into PEG, dioxane, and sulfonic acid (Zalipsky, (1995), Bioconjugate Chem., 6, 150-165). Epoxides may also been used for creating amine bonds but are much less reactive than the above mentioned groups.

Converting PEG into a chloroformate with phosgene gives rise to carbamate linkages to Lysines. This theme can be played in many variants substituting the chlorine with N-hydroxy succinimide (U.S. Pat. No. 5,122,614, (1992); Zalipsky et al., (1992), Biotechnol. Appl. Biochem., 15, p. 100-114; Monfardini et al., (1995), Bioconjugate Chem., 6, 62-69, with imidazole (Allen et al., (1991), Carbohydr. Res., 213, pp. 309-319), with para-nitrophenol, DMAP (EP 632 082 A1, (1993), Looze, Y.) etc. The derivatives are usually made by reacting the chloroformate with the desired leaving group. All these groups give rise to carbamate linkages to the peptide.

Furthermore, isocyanates and isothiocyanates may be employed yielding ureas and thioureas, respectively.

Amides may be obtained from PEG acids using the same leaving groups as mentioned above and cyclic imid thrones (U.S. Pat. No. 5,349,001 (1994), Greenwald et al.). The reactivity of these compounds is very high but may make the hydrolysis to fast.

PEG succinate made from reaction with succinic anhydride can also be used. The hereby comprised ester group make the conjugate much more susceptible to hydrolysis (U.S. Pat. No. 5,122,614, (1992), Zalipsky). This group may be activated with N-hydroxy succinimide.

Furthermore, a special linker can be introduced. The oldest being cyanuric chloride (Abuchowski et al., (1977), J. Biol. Chem., 252, 3578-3581; U.S. Pat. No. 4,179,337, (1979), Davis et al.; Shafer et al., (1986), J. Polym. Sci. Polym. Chem. Ed., 24, 375-378.

Coupling of PEG to an aromatic amine followed by diazotation yields a very reactive diazonium salt which in situ can be reacted with a peptide. An amide linkage may also be obtained by reacting an azlactone derivative of PEG (U.S. Pat. No. 5,321,095, (1994), Greenwald, R. B.) thus introducing an additional amide linkage.

As some polypeptides do not comprise many Lysines it may be advantageous to attach more than one PEG to the same Lysine. This can be done e.g., by the use of 1,3-diamino-2-propanol.

PEGs may also be attached to the amino-groups of the polypeptide with carbamate linkages (WO 95/11924, Greenwald et al.). Lysine residues may also be used as the backbone.

The coupling technique used in the examples is the N-succinimidyl carbonate conjugaion technique descried in WO 90/13590 (Enzon).

Compositions

The present invention also relates to a composition comprising a variant of the invention and optionally a pharmaceutically acceptable carrier and/or adjuvant and a method for preparing such a composition comprising admixing the variant of the invention with an acceptable pharmaceutical carrier and/or adjuvant. In a particular embodiment the composition is suitable for treating an immunological disorder, such as allergy in animals or humans, such as a vaccine.

Pharmaceutical carriers and/or adjuvants includes saline, glycerol, aluminium hydroxide, aluminium phosphate, calcium phosphate, saponins (e.g., Q21 and Quill A), squalene based emulsions (e.g., MF59), monophosphoryl lipid A (and synthetic mimics), polylactide co-glycolid (PLG) particles, ISCOMS, liposomes, chitosan, bacterial DNA (e.g., unmethylated CpG containing sequences). Suitable carriers also include pharmaceutically acceptable solvents and/or tabletting aids/auxilliaries.

Use of Vaccination Antigen Polypeptide Variants and Compositions Containing Them

In a further aspect the invention provide use of the variant or the composition of the invention as a medicament, particularly for the treatment of an immunological disorder, such as allergy in animals and humans and/or for the preparation of a medicament for the treatment of such immunological disorder.

Traditionally, allergy vaccination is performed by parenteral, intranasal, or sublingual administration in increasing doses over a fairly long period of time, and results in, so called, desensitisation of the patient. The exact immunological mechanism is not known, but induced differences in the phenotype of allergen specific T and B cells are thought to be of particular importance.

Compared to conventional types of vaccination, allergy vaccination is complicated by the existence of an ongoing immune response in allergic patients. This immune response is characterised by the presence of allergen specific IgE, that will mediate the release of allergic mediators, thereby inducing allergic symptoms upon exposure to allergens. Thus, allergy vaccination using native and/or naturally occurring allergens has an inherent risk of side effects being in the utmost consequence life threatening to the patient.

Approaches to circumvent this problem may be divided in three categories. In practise measures from more than one category are often combined. First category of measures includes the administration of several small and increasing doses over a long period to reach a substantial accumulated dose. The theory being, that the protective immune response slowly is allowed to be initiated, before potentially anaphylactic doses of allergen is administrated. Second category of measures includes physical modification of the allergen by incorporation of the allergen into e.g., a gel formulation such as a aluminium hydroxide. Aluminium hydroxide has an adjuvant effect and a depot effect of slow allergen release, thus reducing the the tissue concentration of the allergen. Third category of measures include as described herein modification of the allergen for the purpose of reducing allergenicity.

The immunotherapeutic effect of an allergy vaccine can be assessed in a number of different ways. One is to measure the specific IgE binding, the reduction of which indicates a better safety profile (however not necessarily a better vaccine potential) (WO 99/47680, ALK-ABELLÓ). Also, several cellular assays could be employed to show the modified immune response indicative of good allergy vaccine potential as shown in several publications, all of which are hereby incorporated by reference (van Neerven et al., “T lymphocyte responses to allergens: Epitope-specificity and clinical relevance”, Immunol Today, 1996, vol. 17, pp. 526-532; Hoffmann et al., Allergy, 1999, vol. 54, pp. 446-454, WO 99/07880). Basophil histamine release: e.g., Swoboda et al., Eur. J. Immunol., vol. 32, pp 270-280, 2002.

Also skin prick testing could be employed for example as described in Kronqvist et al Clin Exp Allergy 2000 vol 30 pp 670-676.

Eventually, clinical trials with allergic patients could be employed using cellular or clinical end-point measurements. (Ebner et al., Clin. Exp. All., 1997, Vol. 27, pp. 107-1015; Int. Arch. Allergy Immunol., 1999, Vol. 119, pp. 1-5).

### EXAMPLES

Methods

Sandwich ELISA

Immunoplates (Nunc Maxisorb; Nunc-Nalgene) are coated overnight at 4 degree C. with at suitable dose of polyclonal rabbit anti Der p 1 antibody. The plates are then washed thoroughly with 0.15 M Phosphate Buffered Saline (PBS) containing 0.05% Tween 20 (PBST), and remaining binding sites are blocked with PBS with 2% skim milk powder, 1 h at room temperature. Samples, it can be purified, semi-purified recombinant group 1 mite polypeptide variant allergen or crude culture broth containing protein of interest, are added in a suitable dose or dose-range. The plates are then washed thoroughly with 0.15 M PBST before the allergens are detected by incubation with biotinylated monoclonal anti Der p 1 antibody (INDOOR) 1 h at room temperature. Wash again in 0.15 M PBST. Conjugate with complexes of Streptavidin:Horse Radish Peroxidase (Kierkegaard & Perry) for 1 h at room temperature. Repeat washing step and develop by adding 3,3′,5,5′-tetramethylbenzidine hydrogen peroxide (TMB Plus, Kem-En-Tec) and stop reaction by addition of 0.2 M H2SO4. OD450 will reflex allergen binding to the immunoglobin, and it is thus possible to detect and also determine the amount of allergen bound if natural Der p 1 (available from Indoor biotechnologies, product number: NA-DP1) in known concentrations is included in the experiment in a dose rage.

### Example 1

### Finding of Epitope Patterns Within Oligo Peptides with Antibody Reactivity

A high diversity library of phages expressing random oligomeric peptides (hexa, hepta, octa, nona and/or dodeca peptides) as part of their surface proteins, were screened for their capacity to bind antibodies. The phage libraries were obtained from Schafer-N, Copenhagen, Denmark.

Antibody samples were raised in animals (Rat, Rabbits or Mice) by parenteral or mucosal administration of each of the proteins listed below. The antibodies were dissolved in phosphate buffered saline (PBS). In some cases, antibodies of specific subclasses were purified from the serum of immunised animals by capryilic acid precipitation (for total IgG) or by affinity chromatography using paramagnetic immunobeads (Dynal AS) loaded with one of the following antibodies: mouse anti-rat IgG1 or rat anti-mouse IgE.
1) amylase M560 from Bacillus sp. DSM 12649, (Rat IgG)
2) alpha-amylase of Bacillus halmapalus (WO96/23873), which is also called amylase SP722, (Rat IgG)
3) a variant of SP722 with residues 183 and 184 deleted, called JE-1 (WO96/23873), (Rat IgG and Rabbit IgG)
4) Mycelioptora thermopila laccase (WO 95/33836) (Rabbit IgG),
5) T. lanuginosus lipase (Lipolase™) (Rat IgG and Rabbit IgG),
6) family 45 cellulase from Humicola insolens (Carezyme™) (Rabbit IgG),
7) Bacillus lentus protease (Savinase™) (Rat IgG, Mouse IgG, Mouse IgE, and Rabbit IgG),
8) Subtilisin Novo (BPN′) from B. amyloliquefaciens (Rat IgG),
9) The Y217L variant of Subtilisin Novo (Rat IgG),
10) Subtilisin Carlsberg (Alcalase™) (Rat IgG),
11) TY145 protease (Rat IgG),
12) CDJ31 protease,
13) Subtilisin 147 (Esperase™) (Rat IgG),
14) Bacillolysin from Bacillus amyloliquefaciens (Neutrase™) (Rat IgG and Rat IgG1), and
15) Subtilisin PD498 (WO 93/24623) (Rat IgG and Rabbit IgG).

The phage libraries were incubated with the antibody coated beads. For example, phages expressing oligo-peptides with affinity for mouse IgE antibodies were captured onto rat anti-mouse IgE-coated beads and collected by exposing these paramagnetic beads to a magnetic field. The collected phages were eluted from the immobilized antibodies by mild acid treatment, or by elution with intact protein antigen specific for the respective antibody sample (e.g., Savinase for anti-Savinase antibodies). The isolated phages were amplified using methods known in the art. Alternatively, immobilised phages were directly incubated with E. coli for infection. In short, F-factor positive E. coli (e.g., XL-1 Blue, JM101, TG1) were infected with M13-derived vector in the presence of a helper phage (e.g., M13K07), and incubated, typically in 2xYT containing glucose or IPTG, and appropriate antibiotics for selection. Finally, cells were removed by centrifugation. This cycle of events was repeated on the respective cell supernatants, minimum 2 times and maximum 5 times. After selection round 2, 3, 4 and 5, a fraction of the infected E. coli was incubated on selective 2xYT agar plates, and the specificity of the emerging phages was assessed immunologically: Phages were transferred to a nitrocellulase (NC) membrane. For each plate, 2 NC-replicas were made. One replica was incubated with the selection antibodies, the other replica was incubated with the selection antibodies and the immunogen used to obtain the antibodies as competitor. Those plaques that were absent in the presence of immunogen, were considered specific, and were am-plified according to the procedure described above.

The specific phage-clones were isolated from the cell supernatant by centrifugation in the presence of polyethylenglycol. DNA was isolated, the DNA sequence coding for the oligopeptide was amplified by PCR, and its DNA sequence was determined, all according to standard procedures known in the art. The amino acid sequence of the corresponding oligopeptide was deduced from the DNA sequence.

These 256 experimentally determined reactive peptides were supplemented with information on 402 reactive peptides published in the literature:
Allergy 38 (1983) 449-459;
Allergy 56 (2001) 118-125;
Allergy 56 s67 (2001) 48-51;
Allergy 54 (1999) 1048-1057;
Arch Biochem Biophys 342 (1997) 244-253;
B. B. Res. Com. 219 (1996) 290-293;
Biochem J 293 (1993) 625-632;
Bioinformatics 18 (2002) 1358-1364;
Clin Exp Allergy 24 (1994) 100-108;
Clin Exp Allergy 24 (1994) 250-256;
Clin Exp Allergy 31 (2001) 331-341;
Clin Exp Med 24 (1994) 100-108;
Eur J Biochem 245 (1997) 334-339;
Int Arch Allergy Appl Immunol 89 (1989) 342-348;
Int Arch Allergy Appl Immunol 89 (1989) 410-415;
Int Arch Appl Immunol 103 (1994) 357-364;
Int Arch Appl Immunol 92 (1990) 30-38;
J Allergy Clin Immunol 106 (2000) 150-158;
J Allergy Clin Immunol 107 (2001) 1069-1076;
J Allergy Clin Immunol 93 (1994) 3443;
J Biol Chem 271 (1996) 29915-29921;
J Clin Invest 103 (1999) 535-542;
J Immunol 121 (1989) 275-280;
J Immunol 133 (1984) 2668-2673;
J Immunol 140 (1988) 611-616;
J Immunol 147 (1991) 205-211;
J Immunol 151 (1993) 5354-5363;
J Immunol 151 (1993) 7206-7213;
J Immunol Methods 213 (1998) 1-17;
Mol Immunol 25 (1988) 355-365;
Mol Immunol 28 (1991) 1225-1232;
Mol Immunol 29 (1992) 1383-1389;
Mol Immunol 29 (1992) 257-262;
Mol Immunol 30 (1993) 183-189;
Mol Immunol 35 (1998) 293-305;
Mol Immunol 37 (2000) 789-798;
Peptides 21 (2000) 589-599;
Protein Scieince 8 (1999) 760-770;
Scand J Immunol 27 (1988) 587-591;
Science 233 (1986) 747-753;
WO 90/11293;
WO 99/38978;
WO 01/34186;
WO 01/39799;
WO 01/39799;
WO 01/49834;
www.csl.gov.uk/allergen

Thus, in total 658 peptide sequences with specificity for the protein-specific antibodies, described above, were obtained. These sequences were collected in a database, and analysed by sequence alignment to identify epitope patterns observing that conservative alternatives were considered equal (as described above).

Identifying Epitope Patterns

In principle, each of the the 658 reactive (oligo)peptide sequences represented an epitope pattern. However, in the 658 reactive (oligo)peptide sequences some epitope patterns were redundant and to remove redundency among the epitope patterns, the reactive (oligo)peptides sequences were subjected to computerised data analysis.

First all possible dipeptides were generated korresponding to 13² different combinations taking conservative alternatives into account. The presence and frequency of each dipeptide among the 658 reactive (oligo)peptide sequences were listed. Next all possible tripeptides were generated coresponding to 13³ different combinations and again the presence and frequency of each tripeptide among the 658 reactive (oligo)peptide sequences were listed. All possible combinations of the listed dipeptides and tripeptides were then generated including those containing 1, 2, 3 or 4 residues inserted bewteen the dipeptides and tripeptides, these residues selected among the 13 possible residue types. This procedure generated a list of different peptide combinations of 5 to 9 amino acids each containing at least one dipeptide and at lest one tripeptide from the initial listings as well as 0 to 4 residues in between. The frequency of each peptide combination among the 658 reactive (oligo)peptide sequences were then ranked and relevant epitope patterns were selected by a procedure where reactive peptides covered by the most frequent combination were first selected and separated from the group of the 658 reactive peptides. Then reactive peptides covered by the second most frequent combination were selected and separated from the remaining group. Then reactive peptides covered by the third most frequent combination were selected and separated from the remaining group. This procedure was repeated until combinations covering all 658 reactive peptides are found. This way it was found that 357 combinations (epitope patterns) were found to cover all the 658 reactive peptides.

### Example 2

### Predicting Epitopes

The Der p 1 model was built using the following three-dimensional structures as templates:

| PDB entry | Protein |
|---|---|
| 9PAP | Papaya papain |
| 2ACT | Kiwi actinidin |
| 1PPO | Papaya omega protease |

The sequences of the three templates and mature Der p 1 were aligned using ClustalW 1.7 (as described in Higgins et al., Methods Enzymol., vol. 266, pp 383-402, 1996, and Thompson, et al., Nucleic Acids Research, vol. 22, pp 4673-4680, 1994. The alignment is shown below. As the first 9 residues of mature Der p 1 have no corresponding residues in any of the templates, reliable modelling of these residues is not possible, and hence, the 9 N-terminal residues are not included in the Der p 1 model.

The “Modeler 5.0” program (Accelrys Software, San Diego, Calif., USA) was used to build the three-dimensional model of Der p 1. “Modeler 5.0” was started from the “InsightII” molecular modelling software (Accelrys Software, San Diego, Calif., USA) using the following parameters: Number of models: 1, Optimize level: None, More options: Yes, Optimize loop: Yes, Number of loop models: 2, Loop optimite level: Low, Build hydrogens: None.

Using “InsightII”, hydrogens were added and CHARMm potentials and partial charges assigned to the model. Using “CHARMm” (Accelrys Software, San Diego, Calif., USA), 100 steps of ABNR mimimization were applied to relax the model.

Sequence Alignment:

| >P1;P_1PPO | | |
|---|---|---|
| structureX:P_1PPO:1 | : :216 | : :unknown:unknown:−1.00:−1.00 |
| ---------LPENVDWRKKGAVTPVRHQGSCGSCWAFSAVATVEGINKIRTGKLVELSEQELVDCER--RSHGCK | | |
| GGYPPYALEYVAKNG-IHLRSKYPYKAKQGTCRAKQVGGPIVKTSGVGRVQPNNEGNLLNAIAKQPVSVVVES-- | | |
| -KGRPFQLYKGGIFEGPCG--TKVDHAVTAVGYGKSGGKGYILIKNSWGTAWGEKGYIRIKRAPGNSPGVCGLYK | | |
| SSYYPTKN* | | |
| >P1;P_2ACT | | |
| structureX:P_2ACT:1 | : :218 | : :unknown:unknown:−1.00:−1.00 |
| ---------LPSYVDWRSAGAVVDIKSQGECGGCWAFSAIATVEGINKITSGSLISLSEQELIDCGRTQNTRGCD | | |
| GGYITDGFQFIINDGGINTEENYPYTAQDGDCDVALQDQKYVTIDTYENVPYNNEWALQTAVTYQPVSVALDA-- | | |
| -AGDAFKQYASGIFTGPCG--TAVDHAIVIVGYGTEGGVDYWIVKNSWDTTWGEEGYMRILRNVGG-AGTCGIAT | | |
| MPSYPVKY* | | |
| >P1;P_9PAP | | |
| structureX:P_9PAP:1 | : :212 | : :unknown:unknown:−1.00:−1.00 |

| ---------IPEYVDWRQKGAVTPVKNQGSCGSCWAFSAVVTIEGIIKIRTGNLNQYSEQELLDCDR--RSYGCN | | |
|---|---|---|
| GGYPWSALQLVAQYG-IHYRNTYPYEGVQRYCRSREKGPYAAKTDGVRQVQPYNQGALLYSIANQPVSVVLQA-- | | |
| -AGKDFQLYRGGIFVGPCG--NKVDHAVAAVGYGPN----YILIKNSWGTGWGENGYIRIKRGTGNSYGVCGLYT | | |
| SSFYPVKN* | | |
| >P1;DER_P_1_MATURE | | |
| sequence:DER_P_1_MATURE:1 | : :222 | : :unknown:unknown:−1.00:−1.00 |

TNACSINGNAPAEIDLRQMRTVTPIRMQGGCGSCWAFSGVAATESAYLAYRNQSLDLAEQELVDCAS---QHGCH
GDTIPRGIEYIQHNG-VVQESYYRYVAREQSCR--RPNAQRFGISNYCQIYPPNVNKIREALAQTHSAIAVIIGI
KDLDAFRHYDGRTIIQRDNGYQPNYHAVNIVGYSNAQGVDYWIVRNSWDTNWGDNGYGYFAANIDLMMIEEYPYV
VIL-----*

Surface accesibility was measured for each amino amino acid in SEQ ID NO: 1 using the DSSP program (see W. Kabsch and C. Sander, Biopolymers 22 (1983) 2577-2637) in percent of a standard value for that amino acid. The standard values generated according to established methods by analysing average surface accesibility of an amino acid in a 20-mer homogeneous peptide in helix formation using the DSSP program. For each of the 20 different residues the average surface accessibility was as follows:

| Residue | Accessiblity Å² |
|---|---|
| A | 62 |
| C | 92 |
| D | 69 |
| E | 156 |
| F | 123 |
| G | 50 |
| H | 130 |
| I | 84 |
| K | 174 |
| L | 97 |
| M | 103 |
| N | 85 |
| P | 67 |
| Q | 127 |
| R | 211 |
| S | 64 |
| T | 80 |
| V | 81 |
| W | 126 |
| Y | 104 |

Epitopes were predicted by a computer program on a 3-dimensional model of Der p 1 (SEQ ID NO: 1) by using the epitope patterns found in example 1 as follows:

(1) For all amino acids in SEQ ID NO: 1 it was examined if (a) the amino acid type match the first amino acid of an epitope patterns and (b) the solvent surface accessibility greater than or equal to a predefined value, e.g., 20%. Those amino acid satisfying 1(a) and 1(b) are selected.

(2) For all amino acids within a distance of 10 Å from the amino acids selected in step 1 it is examined if (a) the amino acid type matches the second amino acid of the pattern and (b) the surface accessibility greater than or equal to the predefined value, e.g., 20%. Those amino acid satisfying 2(a) and 2(b) are selected.

(3) For all amino acids within a distance 10 Å from the amino acids selected in step 2 it is examined if (a) the amino acid type matches the third amino acid of the pattern and (b) the surface accessibility greater than or equal to the predefined value, e.g., 20%. Those amino acid satisfying 3(a) and 3(b) are selected.

(4) Repeating step 3 for all amino acids in the epitope pattern.

Further, a limit of 25 Å was set as the maximum distance between any two epitope residues.

This procedure was carried out for all 357 epitope patterns for each of the following settings for surface accessibility cutoff: 30, 40, 50, 60, 70 and 80%. Epitope patterns finding a match on the 3 dimensional structure of SEQ ID NO: 1 according this procedure are predicted as epitopes.

Finally, for each of the seven settings for solvent accessibility, a table of all Der p 1 amino acids was created, in which each amino acid residue was given a score by adding up the number of times it appeared in one of the epitopes (at that solvent setting). This score will be an indication of the likelyhood that modification (substitution, insertion, deletion, glycosylation or chemical conjugation) of that amino acid will, result in a variant of lower antigenicity. All amino acids of the protein can then be ranked according to this score and those with highest scores can be selected for mutagenesis.

The 10% highest scoring amino acids (i.e. the 22 highest scoring ones), at each solvent accessibility setting, are shown in the table below (however for solvent accessibility settings of 70 and 80%, less than 22 residues scored at all, so in those two cases all residues that scored are listed). Cysteines are omitted, as they are often involved in maintaining three-dimensional structure.

| Minimum Solvent | |
|---|---|
| Accessibility | Top scoring 10% of amino acids |
| 20% | G32, A46, Y47, L55, D64, A66, S67, |
| | G73, T75, I80, Q84, N86, G87, A98, R105, |
| | Q109, R110, F111, G112, I113 |
| 30% | G30, G32, D64, A66, S67, Q84, N86, |
| | G87, Y93, A98, R99, Q101, R105, |
| | P106, F111, G112, I113, I158, A205 |
| 40% | G29, G30, G32, L55, A66, S67, Q84, |
| | S92, Y93, Y96, A98, R99, E100, |
| | Q101, R104, Q109, F111, I113, I144, |
| | K145, D146, N163 |
| 50% | G29, G30, G32, S67, R99, E100, I144, |
| | K145, D146, 0148, R151, I159, Q160, |
| | R161, D162, N163, G164, Y165, Q166, |
| | A180, V183, D184 |
| 60% | A10, A12, E13, A132, I144, K145, D146, |
| | D148, R151, I159, Q160, R161, D162, |
| | N163, G164, Y165, Q166, N179, A180, |
| | G182, D184, I208 |
| 70% | A10, A12, I144, K145, D146, D148, I159, |
| | R161, G164, Y165, Q166, D184, I208 |
| 80% | D146, D148, G164, Y165, Q166 |

From this procedure it was found that residues A10, A12, E13, G29, G30, G32, A46, Y47, L55, D64, A66, S67, G73, T75, I80, Q84, N86, G87, S92, Y93, Y96, A98, R99, E100, Q101, R104, R105, P106, Q109, R110, F111, G112, I113, A132, I144, K145, D146, D148, R151, I158, I159, Q160, R161, D162, N163, G164, Y165, Q166, N179, A180, G182, V183, D184, A205, I208 of SEQ ID NO: 1, each belonged to the top 10% highest ranking residues at at least one solvent accesibility setting.

Residues A10, A12, G30, G32, A46, Y47, L55, D64, A66, S67, G87, S92, A98, R99, E100, Q101, R105, R110, F111, G112, I113, I144, K145, D146, D148, R151, I159, Q160, R161, D162, N163, G164, Y165, Q166 of SEQ ID NO: 1, each belonged to the top 5% highest ranking residues at at least one solvent accesibility setting.

By studying the positions of the top 10% scoring amino acids on the three-dimensional model of Der p 1, it is possible to define 5 epitope containing regions on the molecule. The 50% highest scoring amino acids within each region are then selected as best candidates for mutation: Residues A10, A12, G32, L55, A66, S67, G87, A98, R99, F111, G112, I113, I144, D146, D148, I159, R161, G164, Q166, A180, D184, A205 and I208 of SEQ ID NO: 1.

### Example 3

### Aligning Group 1 Mite Polypeptides

The sequences of five different native group 1 mite prepro-polypeptides were aligned:

| | −90 −80 −70 −60 −50 −40 |
|---|---|
| | --+----+----+----+----+----+----+----+----+----+----+----+-- |
| Eur_m1 | MKIILAIASLLVLSAVYARPASIKTFEEFKKAFNKTYATPEKEEVARKNFLESLKYVESN |
| Der_f1 | MKFVLAIASLLVLSTVYARPASIKTFEEFKKAFNKNYATVEEEEVARKNFLESLKYVEAN |
| Der_p1 | MKIVLAIASLLALSAVYARPSSIKTFEEYKKAFNKSYATFEDEEAARKNFLESVKYVQSN |
| Blo_t1 | ------------------------------------------------------------ |
| Der_m1 | ------------------------------------------------------------ |
| | −30 −20 −10 0 10 20 |
| | --+----+----+----+----+----+----+----+----+-----+----+----+- |
| Eur_m1 | KGAINHLSDLSLDEFKNQFLMNANAFEQLKTQFDLNAETYACSINSVSLPSELDLRSLRT |
| Der_f1 | KGAINHLSDLSLDEFKNRYLMSAEAFEQLKTQFDLNAETSACRINSVNVPSELDLRSLRT |
| Der_p1 | GGAINHLSDLSLDEFKNRFLMSAEAFEHLKTQFDLNAETNACSIN-GNAPAEIDLRQMRT |
| Blo_t1 | ------------------------------------------------IPANFDWRQKTH |
| Der_m1 | --------------------------------------TQACRINSGNVPSELDLRSLRT |
| | 30 40 50 60 70 |
| | ---+----+----+----+----+----+----+----+----+------------+--- |
| Eur_m1 | VTPIRMQGGCGSCWAFSGVASTESAYLAYRNMSLDLAEQELVDCASQN--------GCHG |
| Der_f1 | VTPIRMQGGCGSCWAFSGVAATESAYLAYRNTSLDLSEQELVDCASQH--------GCHG |
| Der_p1 | VTPIRMQGGCGSCWAFSGVAATESAYLAYRNQSLDLAEQELVDCASQH--------GCHG |
| Blo_t1 | VNPIRNQGGCGSCWAFAASSVAETLYAIHRHQNIILSEQELLDCTYHLYDPTYKCHGCQS |
| Der_m1 | VTPIRMQG---------------------------------------------------- |
| | 80 90 100 110 120 130 |
| | --+----+----+----+----+----+----+----+----+----+----+----+-- |
| Eur_m1 | DTIPRGIEYIQQNGVVQEHYYPYVAREQSCHR-PNAQRYGLKNYCQISPPDSNKIRQALT |
| Der_f1 | DTIPRGIEYIQQNGVVEERSYPYVAREQRCRR-PNSQHYGISNYCQIYPPDVKQIREALT |
| Der_p1 | DTIPRGIEYIQHNGVVQESYYRYVAREQSCRR-PNAQRFGISNYCQIYPPNVNKIREALA |
| Blo_t1 | GMSPEAFKYMKQKGLLEESHYPYKMKLNQCQANARGTRYHVSSYNSLRYRAGDQEIQAAI |
| Der_m1 | ------------------------------------------------------------ |
| | 140 150 160 170 180 190 |
| | --+----+----+----+----+----+----+----+----+----+----+----+-- |
| Eur_m1 | QTHTAVAVIIGIKDLNAFRHYDGRTIMQHDNGYQPNYHAVNIVGYGNTQGVDYWIVRNSW |
| Der_f1 | QTHTAIAVIIGIKDLRAFQHYDGRTIIQHDNGYQPNYHAVNIVGYGSTQGDDYWIVRNSW |
| Der_p1 | QTHSAIAVIIGIKDLDAFRHYDGRTIIQRDNGYQPNYHAVNIVGYSNAQGVDYWIVRNSW |
| Blo_t1 | MNHGPVVIYIHGTEA-HFRNLRKGILRGAGYNDAQIDHAVVLVGWGTQNGIDYWIVRTSW |
| Der_m1 | ------------------------------------------------------------ |
| | 200 210 220 |
| | --+----+----+----+----+----+-- |
| Eur_m1 | DTTWGDNGYGYFAANINLMMIEQYPYVVML |
| Der_f1 | DTTWGDSGYGYFQAGNNLMMIEQYPYVVIM |
| Der_p1 | DTNWGDNGYGYFAANIDLMMIEEYPYVVIL |
| Blo_t1 | GTQWGDAGYGFVERHHNSLGINNYPIYASL |
| Der_m1 | ------------------------------ |

Der p 1 holds an 18 amino acids signal peptide and an 80 amino acids propeptide while the mature Der p 1 is a 222 amino acid molecoule. In the alignement a gap has been made in position 8 of Der p 1 because it lacks an amino acid here compared to other group 1 mite polypeptides. Similar descriptions may be made for Eur m1, while for Der f 1 only the mature polypeptide is shown and for Der m 1 only a fraction of the sequence has been identified.

The alignment confirms high homology among group 1 mite polypeptides and exceptionally high homology or conservatism in the amino acids identified as involved in epitopes. Thus it seems safe to say that the epitopes and thus mutations suggested for Der p 1 are also suitable for the remaining members of group 1 mite polypeptides.

### Example 4

### Construction and Expression of Enzyme Variants

Der p 1 variants of the invention comprising specific substitutions were made by cloning of DNA fragments (Sambrook et al., Molecular Cloning: A Laboratory Manual, 2nd Ed., Cold Spring Harbor, 1989) produced by PCR with oligos containing the desired mutations.

Recombinant Der p 1 and all variants were expressed with the Der p 1 propeptide on and they all had the mutation S54N which disrupts the only N-glycosylation motif within the mature sequence.

The template plasmid DNA may be pSteD212, or an analogue of this containing Der p 1 or a variant of Der p 1. Mutations were introduced by oligo directed mutagenesis to the construction of variants. The Der p 1 plasmid constructs were transformed into S. cerevisiae, strain JG169, as described by Becker and Guarente (1991, Methods Enzymology, 194: 182-187).

The Cystein protease or variants hereof of the present invention were located in vector pSteD212, which is derived from yeast expression vector pYES 2.0 (Kofod et al. 1994 J. Biol. Chem. 269: 29182-29189 and Christgau et al. 1994, Biochem. Mol. Biol. Int. 33: 917-925).

This plasmid replicated both in E. coli and in S. cerevisiae. In S. cerevisiae Der p 1 or variants hereof according to the invention were expressed from this plasmid.

### Example 5

### Fermentation

Fermentations for the production of Der p 1 enzyme/Der p 1 variants were performed at degree C. on a rotary shaking table (250 r.p.m.) in 500 ml baffled Erlenmeyer flasks containing 100 ml SC medium for 5 days.

Consequently, in order to make e.g. a 2 liter broth 20 Erlenmeyer flasks were fermented simultaneously.

SC Medium (per liteR):

| | |
|---|---|
| Yeast Nitrogen Base without amino acids | 7.5 g |
| Succinic acid | 11.3 g |
| Casamino acid without vitamine | 5.6 g |
| Tryptophan | 0.1 g |

Add H₂O. Autoclave and cool before adding glucose and L-threonin to a final concentration of 4% and 0.02%, respectively.

For agar plates, 20 g bactoagar was added to the medium before autoclave.

### Example 6

### Screening for Der p 1 Variants

For screening of yeast transformants expressing Der p 1 or Der p 1 variants, the transformation solution was plated on SC-agar plates for colony formation at 30 degree C., 3 days. Colonies were inoculated in 96 micro-well plates, each well containing 200 microL SC medium. The plates were fermented at 30 degree C., 250 r.p.m. for 5 days.

50 microL culture broth was diluted 1:1 in 0.15 M Phosphate Buffered Saline (PBS) before OD450 measurement in sandwich ELISA. Culture broth of yeast transformed with a plasmid without the Der p 1 gene was used as background with an average OD450 of 0.55. Variants tested in sandwich ELISA with OD450>0.55 were sequenced and approved as correctly folded and yeast expressed Der p 1 variants.

A part of the variants were concentration determined directly in culture broth by the sandwich ELISA technique with natural Der p 1 as a standard.

Der p 1 variants identified and determined by both OD450>0.55 and in a quantitative sandwich ELISA (concentrations given as microg/mL) are shown in table below.

| Variant no. | Mutations | |
|---|---|---|
| | | concentration |
| DP003 | S54N | 11 |
| DP009 | G32E, S54N, A66V | 0.2 |
| DP010 | G32D, S54N, A66V | 2.9 |
| DP017 | S54N, G87V | 0.016 |
| DP018 | S54N, G87E, I113E | 0.032 |
| DP019 | S54N, I113V | 7.05 |
| DP020 | S54N, G87E, I113V | 0.032 |
| DP021 | S54N, I113D | 0.032 |
| DP024 | S54N, T75D | 5.4 |
| DP025 | S54N, T75A | 8 |
| DP026 | S54N, T75V | 7.3 |
| DP033 | S54N, D184A | 1.62 |
| DP034 | S54N, D184A, A205D | 0.027 |
| DP035 | S54N, A205D | 0.048 |
| DP049 | A10Y, A12Y, S54N | 0.81 |
| DP050 | A10N, A12N, S54N | 0.027 |
| DP054 | S54N, I159V | 0.97 |
| DP065 | S54N, R99G | 3.9 |
| DP067 | S54N, I144L | 0.1 |
| DP068 | S54N, R161S | 0.1 |
| DP070 | S54N, F111I | 0.6 |
| DP071 | S54N, G87V, G112D | 1.5 |
| | | OD450 > 0.55 |
| DP073 | G32V, S54N, A66V, S67C, A98G | 1.20 |
| DP074 | G32D, S54N, A98V, I144T | 1.01 |
| DP075 | S54N, D64N, A66D | 0.66 |
| DP077 | A66D, R99G | 0.72 |
| DP078 | G32V, S54N, A66G, A98V, R99D | 1.15 |
| DP079 | S54N, S67H, A98V | 0.69 |
| DP080 | S54N, I144L, D148Y | 0.64 |
| DP081 | S54N, I144L, D148N, R161S, G164D | 1.10 |
| DP082 | S54N, D148N, I159L, R161G, G164D, | 0.62 |
| | Q166L | |
| DP083 | S54N, D148Y, R161S, G164V | 0.62 |
| DP084 | S54N, I144V, D148Y, R161G | 1.19 |
| DP085 | S54N, D148N, R161S, G164V | 1.53 |
| DP086 | S54N, D148H, I159F, R161S, Q166R | 1.16 |
| DP087 | S54N, D148H | 0.66 |
| DP088 | S54N, A180V | 1.12 |
| DP089 | S54N, D184H | 1.27 |
| DP090 | S54N, A180G | 1.30 |
| DP091 | S54N, A180V, D184H | 0.65 |
| DP092 | S54N, A180G, D184N | 0.67 |
| DP094 | S54N, L55V, F111W, G112D | 1.15 |
| DP095 | S54N, G112V, I113L | 1.24 |
| DP096 | S54N, F111I, G112D | 1.36 |

### Example 7

### Purification Method for Pro Form and Mature Form of Der P1 Antigen

Assay for Detection of Der P1 and Pro-Der P1

Qualitative ELISA (Enzyme linked immunosorbent assay) for detection of Der P1 and Pro-Derp1.

Ployclonal antibodies were raised in Rabbits against Native Der P1 bought from Indoor technologies. The polyconal antibodies were purified by ammonium sulphate precipitation and on Protein a column as described in literature and finally dialyzed against 50 mM Borate pH 8 buffer.

The purified antibodies against Der P1 were labelled with Biotin using NHS-Biotin as described in Product sheet described by Pierce Chemicals 3747 N. Meridian Rd. PO Box 117. Rockford, Ill. 61105 USA, and the labelled antibodies were used as detecting antibodies.

Method for fast qualitative detection of Der P1 or Pro Der P1 was as follows.

Immunosrop microtiter plates were bought from NUNC and microtiter wells were coated with 100 microliters of 10 microgram per ml unlabelled polyclonal antibodies against Der P1 for overnight at 4 degree centigrade. The microtiter wells were then washed with PBS Tween buffer as described in literature. Microtiter wells were then saturated with 200 microliters of PBS buffer containing 10 milligrams per milliliters BSA and 0.05% Tween 20 and incubated for 30 minutes at room temperature.

Microtiter wells were then washed thrice with PBS buffer containing 0.05% Tween 20.

Microtiter wells were then coated with 100 microliters fractions containing Der P1 or Pro-Der P1 and incubated for 20 minutes with gentle shaking. Microtiter wells were then washed thrice with PBS buffer containing 0.05% Tween 20. Microtiter wells were then coated with 100 microliters of biotin labelled polyclonal antibodies around 1 microgram per milliliters diluted in PBS buffer with 0.05% Tween 20 and incubated for 20 minutes at room temperature with gentle shaking.

Microtiter wells were then washed thrice with PBS buffer and coated with 100 microliters of properly diluted Immunopure Avidin Horse radish peroxidase conjugate which was purchased from Pierce chemicals. After 20 minutes incubation wait room temperature the wells were then washed with PBS buffer containing 0.05% Tween 20.

One hundred microliters of Horse Radish peroxidase substrate TMB One purchased form Kem EN Tec was then added to the microtiter wells and incubated for few minutes and reaction was stopped by adding Phosphoric acid as described by KEM EN TEC. For blank exact same procedure was carried out but no antigen was included in the wells.

This method can be used as qualitative assay for detection of Der P1 or Pro Der P1.

Method for Purification of Der P1 and Pro Der P1.

One liter fermentation supernatant of Pro form of Der P1 antigen (Dermatophagoides pteronyssinus) expressed in Yeast or A. oryzae was centrifuged and precipitate containing cell debris was discarded. The cell supernatants were then sterile filtered under pressure through 0.22μ sterile filter Seitz-EKS obtained from Pall Corporation (Pall Seitz Schenk Filter system GmbH Pianiger Str. 137D Cad Kreuznach Germany).

Sterile filtered cell supernatant containing the desired protein was then concentrated using Ultra filtration technique using 10 kDa cut off membrane purchased from from Millipore Corporation, Bedford, Mass. 01730 USA: The small molecules under 10 kDa were then removed by dia filtration using 50 mM Borate pH 8 as buffer.

To the concentrated and dia filtrated supernatant containing the desired protein solid ammonium sulphate was gradually added under gentle stirring to a final concentration of one M ammonium sulphate and pH was adjusted to 8.

Hydrophobic interaction chromatography was carried out on 50 ml XK26 column purchased from Amersham-Pharmacia which was packed with Toyopearl Phenyl-650 matrix purchased from TOSOH Bioscience GmbH Zettacchring 6, 70567 Stuttgart, Germany.

The column was washed then equilibrated with 1 M ammonium sulphate dissolved in 50 mM Borate pH 8.

The concentrated fermentation supernatant was then applied on the column with a flow of 20 mL per minute. Unbound material was then washed out using 1 M ammonium sulphate dissolved in the borate pH 8 buffer (Buffer A). When all the unbound material was washed out from the column which was monitored using UV detector attached to fraction collector from Amesham Pharmacia.

Bound proteins were then eluted with buffer B which contained 50 mM Borate pH 8 without any other salt and 10 ml fractions were collected. Fractions contain desired protein were checked by SDS-PAGE. Fractions containing Protein with molecular weights between 33 kDa and 22 kDa and found immunoreactive in the qualitative as described above were then pooled and further purified on anion exchange chromatography.

Anion Exchange Chromatography of Der P1 and Pro Der P1

Anion exchanger fast flow Q sepharose 50 ml column XK26 pre-packed by Amersham Pharmacia was washed and equilibrated with 50 mM Borate pH 8 buffer.

Pool containing Der P1 and or Pro Der P1 from Hydrophobic chromatography was then diluted to adjust ionic strength below 4 mSi and pH was adjusted to 8. The diluted pool was then applied on the Fast flow Q sepharose column with flow rate 20 ml per minute and unbound material was washed with the 50 mM Borate buffer pH 8 as buffer A.

Bound proteins were then eluted with linear gradient using buffer B containing 50 mM Borate pH 8 with 1 M salt as Sodium chloride. Total buffer used was 20 column volumes.

All the fractions were then analysed by SDS-PAGE and qualitative ELISA assay.

Proteins with molecular weight around 30 kDa were then pooled as Pro-Der P1 and mature Der P1 due to slight processing was observed as 20 kDa Protein. The purified proteins were then analysed for N-terminal after SDS-PAGE and blotting on PVDF membrane by Using applied Bio system equipment.

### Example 8

### In Vivo Assessment of Allergenicity of an Enzyme Variant (MINT Assay)

Mouse intranasal (MINT) model (Robinson et al., Fund. Appl. Toxicol. Vol. 34, pp. 15-24, 1996) can be used to verify allergenicity of group 1 mite polypeptide variants.

Mice are dosed intranasally with the group 1 mite polypeptide variant on the first and third day of the experiment and from thereon on a weekly basis for a period of 6 weeks. Blood samples are taken 15, 31 and 45 days after the start of the study, and the serum can subsequently be analysed for IgE levels.

Measurement of the Concentration of Specific IgE in Mouse Serum by ELISA:

The relative concentrations of specific IgE antibodies in serum samples from mice are measured by a three layer sandwich ELISA according to the following procedure:

1) The ELISA-plate (Nunc Maxisorp) is coated with 100 microliter/well rat anti-mouse IgE Heavy chain (HD-212-85-IgE3 diluted 1:100 in 0.05 M Carbonate buffer pH 9.6). Incubated over night at 4° C.

2) The wells are emptied and blocked with 200 microliter/well 2% skim milk in 0.15 M PBS buffer pH 7.5 for 1 hour at 4° C. The plates are washed as before.

3) The plates are incubated with dilutions of mouse sera (100 microL/well), starting from an 8-fold dilution and 2-fold dilutions hereof in 0.15 M PBS buffer with 0.5% skim milk and 0.05% Tween20. Appropriate dilutions of positive and negative control serum samples plus buffer controls are included. Incubated for 1 hour at room temperature. Gently shaken. The plates are washed 3 times in 0.15 M PBS buffer with 0.05% Tween20.

4) 100 microliter/well of group 1 mite polypeptide variant diluted to 1 microgram protein/ml in 0.15 M PBS buffer with 0.5% skim milk and 0.05% Tween20 is added to the plates. The plates are incubated for 1 hour at 4° C. The plates are washed as before.

5) Specific polyclonal anti-group 1 mite polypeptide variant antiserum serum (pig) for detecting bound antigen is diluted in 0.15 M PBS buffer with 0.15% skim milk and 0.05% Tween20. 100 microL/well and incubated for 1 hour at 4° C. The plates are washed as before.

6) 100 microliter/well pig anti-rabbit Ig conjugated with peroxidase diluted 1:1000 in 0.15 M PBS buffer with 0.5% skim milk and 0.05% Tween20 is added to the plates. Incubated for 1 hour at 4° C. The plates are washed as before.

7) 100 μl/well TMB (Plus (Ready-to-go substrate; Kem-En-Tec, Cat. No.: 4390A) is added, and the reaction is allowed to run for 10 min.

8) The reaction is stopped by adding 100 microliter/well 1 M H₂SO₄.

9) The plates are read at 450 nm with 620 nm as reference.

The dose response curves are graphed, and fitted to a sigmoid curve using non-linear regression, and the EC50 is calculated for the group 1 mite polypeptide variant.

Measurement of the Concentration of Specific IgG1 in Mouse Serum by ELISA

The relative concentrations of specific IgG1 antibodies in serum samples from mice are measured by a three layer sandwich ELISA according to the following procedure:

1) The ELISA-plate (Nunc Maxisorp) is coated with 100 microliter/well of group 1 mite polypeptide variant diluted in PBS to 10 microg/ml. Incubated over night at 4° C.

2) The wells are emptied and blocked with 200 microliter/well 2% skim milk in 0.15 M PBS buffer pH 7.5 for 1 hour at 4° C. The plates are washed 3 times with 0.15 M PBS buffer with 0.05% Tween20.

3) The plates are incubated with dilutions of mouse sera (100 microL/well), starting from an 20-fold dilution and 3-fold dilutions hereof in 0.15 M PBS buffer with 0.5% skim milk and 0.05% Tween20. Appropriate dilutions of positive and negative control serum samples plus buffer controls are included. Incubated for 1 hour at room temperature. Gently shaken. The plates were washed as before.

4) 100 microliter/well biotinylated Rat-anti-mouse IgG, (Serotec, Cat. No.: MCA 336B), diluted 2000× in 0.15 M PBS buffer with 0.5% skim milk and 0.05% Tween20 is added to the plates. The plates are incubated for 1 hour at 4° C. The plates are washed as before.

5) 100 microliter/well Horseradish Peroxidase-conjugated Streptavidin (Kierkegaard & Perry, Cat. No.: 14-30-00), diluted 1000× in 0.15 M PBS buffer with 0.15% skim milk and 0.05% Tween20. Incubated for 1 hour at 4° C. The plates are washed as before.

6) 100 μl/well TMB (Plus (Ready-to-go substrate; Kem-En-Tec, Cat. No.: 4390A) is added, and the reaction is allowed to run for 10 min.

7) The reaction is stopped by adding 100 microliter/well 1 M H₂SO₄.

8) The plates are read at 450 nm with 620 nm as reference.

The dose response curves are plotted and fitted to a sigmoid curve using non-linear regression, and the EC50 is calculated for the group 1 mite polypeptide variant.

### Example 9

### In Vitro Assessment of IgE-Antigenicity of an Enzyme Variant

Reduced IgE binding was verified in vitro by direct or competitive ELISA and Basophil histamine release. Group 1 mite polypeptide variants with reduced IgE-antigenicity can then be tested further in vivo, by skin prick testing.

Direct ELISA:

Immunoplates (Nunc Maxisorp; Nunc-Nalgene) were coated overnight at 4° C. with a suitable dose, or dose-range, of group 1 mite polypeptide allergen or with recombinant group 1 mite polypeptide variant allergen. The plates were then washed thoroughly with Phosphate Buffered Saline (PBS) containing 0.05% Tween 20 (PBST), and remaining binding sites were blocked with PBS containing 2% Skim Milk Powder (SMP). Sera from patients allergic to dust mites, (dust mite allergy was diagnosed on the basis of case history, skin prick testing and determination of specific IgE to dust mite extracts (CAP-RAST measurements)), were then diluted ¼ in PBST and added to the plates and incubated at room temperature (RT) for 1 hour or overnight at 4 degree C. Following a thorough wash with PBST, the allergen-IgE complexes were detected, by incubation with a rabbit anti-human IgE antibody (DakoCytomation), and swine anti-rabbit Ig coupled to horseradish peroxidase. The enzymatic activity was measured by adding TMB from Kem-En-Tec, and the reaction was stopped by adding an equal volume of 0.2 M H2SO4, and quantitaing colour development by measuring optical density at 450 nm (OD450) in an ELISA reader. OD450 reflect IgE binding to the allergen.

First, suitable donors for competitive ELISA were identified as follows: nDer p 1-specific IgE-binding in serum isolated from 23 patients allergic to dust mite allergens were analysed in a dose response curve to nDer p 1 in a direct ELISA. OD450 values obtaining a coating concentration of 500 ng/well allergen were determined, and sera that did not reach an OD450 equal or higher than 0.5 were excluded from further analysis in the competitive ELISA. Sera from donor 1, 3, 6, 7, 9, 13, 14, 22 and 23 were chosen for analysis in competitive ELISA.

TABLE 1
OD450 titer obtaining a coating concentration of 500 ng/well group 1
mite wild type polypeptide was determined in sera isolated from
23 patients allergic to dust mite and are shown in the table below.

| | nDer p 1 specific IgE titer |
|---|---|
| Donor 1 | 0.9741 |
| Donor 2 | 0.1815 |
| Donor 3 | 1.7443 |
| Donor 4 | 0.2672 |
| Donor 5 | 0.2207 |
| Donor 6 | 0.8607 |
| Donor 7 | 1.0819 |
| Donor 8 | 0.2219 |
| Donor 9 | 1.0168 |
| Donor 10 | 0.3519 |
| Donor 11 | 0.4384 |
| Donor 12 | 0.2385 |
| Donor 13 | 1.2593 |
| Donor 14 | 2.3644 |
| Donor 15 | 0.2345 |
| Donor 16 | 0.3490 |
| Donor 17 | 0.1829 |
| Donor 18 | 0.1237 |
| Donor 19 | 0.4820 |
| Donor 20 | 0.1087 |
| Donor 21 | 0.4195 |
| Donor 22 | 1.3897 |
| Donor 23 | 1.9637 |

nDer p 1-specific IgE-binding in serum isolated from 23 patients allergic to dust mite allergens were analysed in a dose response curve to nDer p 1 in a direct ELISA. OD450 values correlating to a coating concentration of 500 ng/well allergen was determined and sera that did not reach an OD450 equal or higher than 0.5 were excluded from further analysis in the competitive ELISA. Based on the data shown in Table x1, sera from donor 1, 3, 6, 7, 9, 13, 14, 22 and 23 were chosen for analysis of the individual variants by competitive ELISA. Data from the full dose-response curve for different variant concentrations confirmed this selection (data not shown).

Competetive ELISA:

Was carried out like direct ELISA, with two exeptions: the immunoplates were coated with a fixed concentration (500 ng/well) of wild type popypeptide, and the diluted serum from allergic patients was pre-incubated with a dose range of group 1 mite wild type polypeptide, recombinant group 1 mite polypeptide wild type or group 1 mite polypeptide variant allergen. When IgE binds to the polypeptide in solution, binding to the platebound wild type polypeptide is reduced, thus reducing the OD450. The reduced IgE binding was interpreted using GraphPad Prism software: OD450 was plotted against the logarithm of dose of variant allergen, thereby creating a sigmodal dose-response curve-fit. By using a model of four-parameter logistic (bottom, top, log EC50 and Hill slope) sigmoidal curve fit in Prism, comparison of EC50 found by incubation with group 1 mite wild type polypeptide and variant allergen was performed. Differences in binding affinity are expressed as an X-fold increase or decrease of the amount of variant required obtaining a 50% inhibition of IgE binding to the group 1 mite wild type polypeptide.

TABLE 2
Dose-response curves in nDer p 1-specific IgE serum isolated from 9
dust mite allergic patients were plotted and fitted to a sigmoid curve,
and the EC50 was calculated for the group 1 mite polypeptide variants.

| | nDer | rec- | rec- | DP | DP | DP | DP | DP | DP | DP |
|---|---|---|---|---|---|---|---|---|---|---|
| donor | p1 | proDerp1 | Derp1 | 019 | 024 | 025 | 026 | 033 | 065 | 071 |
| 1 | 1 | 5 | 2 | 2 | 8 | 2 | 10 | 2 | 3 | 3 |
| 3 | 1 | 2 | 4 | 2 | 11 | 3 | 10 | 3 | 4 | 4 |
| 6 | 1 | 3 | 2 | 1 | 10 | 1 | 5 | 2 | 3 | 3 |
| 7 | 1 | 2 | 2 | 2 | 9 | 1 | 10 | 2 | 4 | 3 |
| 9 | 1 | 2 | 3 | 2 | 10 | 2 | 8 | 2 | 3 | 3 |
| 13 | 1 | 1 | 3 | 2 | 22 | 2 | 39 | 3 | 4 | 4 |
| 14 | 1 | 2 | 1 | 1 | 8 | 1 | 4 | 2 | 2 | 2 |
| 22 | 1 | 1 | 1 | 1 | 9 | 1 | 3 | 2 | 2 | 2 |
| 23 | 1 | 2 | 1 | 1 | 9 | 1 | 9 | 2 | — | 2 |

TABLE 3
Dose response curves in nDer p 1-specific IgE serum isolated from 6
patients with dust-mite allergy were plotted and fitted to a sigmoid curve,
and the EC50 was calculated for group 1 mite polypeptide variants.

| | nDer p 1 | rec-proDerp1 | rec-Derp1 | DP070 |
|---|---|---|---|---|
| Donor 1 | 1 | 1 | 2 | 46 |
| Donor 3 | 1 | 2 | 2 | 43 |
| Donor 7 | 1 | 1 | 2 | 33 |
| Donor 9 | 1 | 1 | 2 | 26 |
| Donor 14 | 1 | 2 | 2 | 23 |
| Donor 23 | 1 | 1 | 2 | 37 |

The data disclosed in Table 2 and Table 3 show that the three variants DP024, DP026, and DP070 have a highly reduced IgE binding as compared to the group 1 mite polypeptide. The mutations introduced in the DP024 variant lowers the affinity for specific serum IgE by a factor of about 8 to 22-fold, the mutations introduced in DP026 lowers the affinity for specific serum IgE by a factor of 3 to 39-fold, and the mutations introduced in the DP070 lowers the affinity for specific serum IgE by a factor of about 23-46, compared to the response of nDer p 1. The data further indicates that the variants DP065 and DP071 have a reduced IgE binding compared to the response of nDer p 1.

Basophil Histamine Release:

Histamine release from basophil leukocytes was performed as follows. Heparinized blood (40 mL) was drawn from each dust-mite allergic patient, stored at room temperature, and used within 24 hours. Twenty-five microliters of heparinized blood was applied to glass fibre coated microtitre wells (Reference Laboratory, Copenhagen, Denmark) and incubated with 25 microliters of a dose-range of wild type polypeptide, recombinant wild type, variant allergen, House Dust mite (HDM) extract or anti-IgE for 1 hour at 37 degree C. All serial-dilutions of allergen were made in PIPES-buffer (Reference Laboratory, Denmark). Thereafter the plates were rinsed with water and interfering substances were removed. Finally, histamine bound to the microfibres was measured spectrophotofluometrically. The results are interpreted using the following formula:
% of Allergen-induced histamine release=(histamine in allergen-stimulated supernatants−basal value)/(total histamine release−basal value)×100,
where
basal value=spontaneous histamine release in supernatants without allergen stimuli, and
total histamine release=total histamine contents in blood sample measured after treatment with perchloric acid 2%.

Specific histamine release greater than 10% was considered as positive.

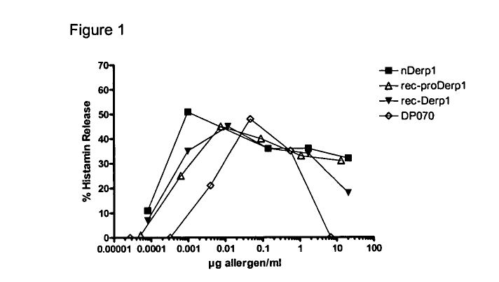

The above procedure was applied on a number of group 1 mite polypeptide variant allergen. % Histamine release as a function of allergen concentration was plotted (see FIG. 1), and the EC50 was determined. Variant allergens with reduced basophile histamine release were selected based upon a shift of the EC50 to higher concentrations as reflecting differences in the induction of histamine release.

Basophile cells from 23 patients allergic to dust-mite and 3 negative donors (negative to histamine release on stimulation with house dust-mite (HDM) extract (ALK-Abello)) were analysed in a histamine release assay on stimulation with group 1 mite polypeptide and group 1 mite polypeptide variants. Of the 23 dust mite allergic patients, only 14 patients were found to induce histamine release in response to stimulation with nDer p 1, demonstrating that approximately 61% of the patients allergic to dust-mite have nDer p 1-specific IgE antibodies (data not shown).

The data disclosed in FIG. 1 shows the potency of rec-proDer p 1, rec-Der p 1 and DP070 variants to induce histamine release in a human basophile cell assay from one dust-mite allergic patient. It is seen that the release curve of DP070 variant is clearly shifted to the right compared to the release curve of nDer p 1, rec-proDer p 1 and rec-Der p 1. The shift indicates that the potency of DP070 variant to induce histamine release is reduced about a 22-fold fold relative to nDer p 1. The shift of the DP070 release curve to the right was found in all the 14 patients with nDer p 1-specific IgE-mediated responses with the corresponding potency reductions ranging from 2 to 22-fold.

Basophile cells from the 9 remaining dust mite allergic patients did not respond to stimulation with concentrations of the group 1 mite polypeptide up to 1.67 μg/ml (data not shown). However, at the highest concentration of group 1 mite polypeptide (20 μg/ml), histamine release was observed from basophile cells from these patients. This induction of histamine release in high concentration of nDer p 1 may be due to low levels of impurities of commercial nDer p 1 and thus, the presence of other dust mite allergens. No histamine release was observed in basophile cells from these 9 patients in response to stimulation with group 1 mite polypeptide variants (data not shown).

Basophile cells from the 3 negative donors did not respond to stimulation with group 1 mite polypeptide or to group 1 mite polypeptide variants, demonstrating no unspecific stimulation of the crude extract.

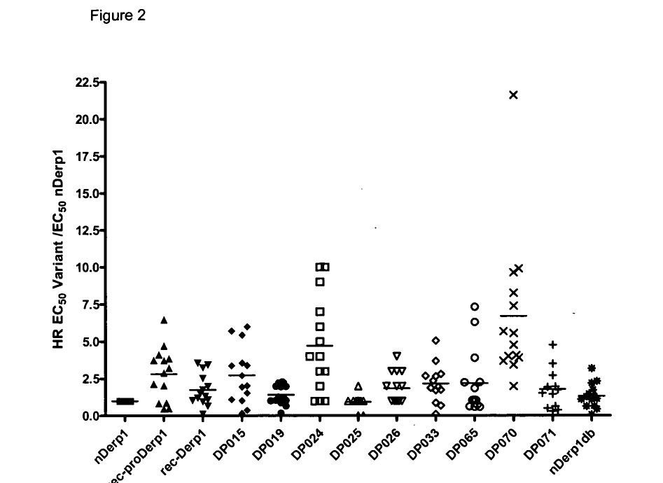

The data disclosed in FIG. 2 demonstrate the overall reduction in IgE antigenicity as measured by histamine release assay. For each variant, in each of the nine patients, the ratio of EC50 value for the variant to the EC50 value of nDer p 1 was calculated. Thus, for the nDer p 1 sample used as control, all donors show a normalized value of 1 (left column). A control series of nDer p 1 samples were included and treated as a normal sample. The result for this series is shown in the rightmost column, and demonstrates a relatively low variability, considering this is a rather sensitive biological response assay. The variants DP024 and DP070 show average improvement factors of around 5 and around 6.7, respectively. Further, the variants DP071, DP065, DP033, and DP026 show improvents in IgE-based antigenicity, as measured by the increase in EC50 value, in most of the donors.

The use of basophil histamine release is described in Nolte H, Schiotz O, Skov PS. A new glass microfibre-based histamine analysis for allergy testing in children. Results compared with conventional leukocyte histamine release assay, skin prick test, bronchial provocation test and RAST. Allergy. 1987 July; 42(5): 366-73; and in: Winther L, Moseholm L, Reimert C M, Stahl Skov P, Kaergaard Poulsen L. Basophil histamine release, IgE, eosinophil counts, ECP, and EPX are related to the severity of symptoms in seasonal allergic rhinitis. Allergy. 1999 May; 54(5): 436-45.

Skin Prick Testing:

Is carried out on patients allergic to group 1 mite polypeptides, and the technique is well known in the art, e.g., Kronquist et al., Clin. Exp. Allergy, 2000, vol. 30, pp. 670-676).

Briefly, 15 microL of solutions containing the recombinant wild type or variant allergens is placed on the patient's forearm. Thereafter, the skin is pricked with a Prick-Lancette (ALK). The test sites are placed 3 cm apart to avoid false-positive results. From an initial solution of recombinant allergens (e.g., 1 mg protein/mL), suitable serial dilutions (e.g., from 100 μg to 0.1 microg/mLI) are made in sterile physiologic saline solution. These dilutions are selected according to the concentration allergens, which elicited significant histamine release by sensitized basophils. It has been shown that the thresholds of positivity for histamine release tests and intradermal reactions are in the same range; and it is assumed that the sensitivity of prick tests is 10² to 10³ times lower than that of intradermal tests. A negative control test is performed with saline solution, and a positive control test is done with histamine at e.g., 1 mg/mL. The diameter of the weal is used as a measure of allergenic reactivity towards that variant, and this allows for comparison of the variant allergens to the parent or native type allergen.

### Example 10

### Assessing Retained Ability to Stimulate T Cells

The lymphocyte fraction from heparinized blood from patients allergic to group 1 mite polypeptides was purified by density gradient centrifugation on Lymphoprep (Axix-Shield PoC, Norway) and resuspended in AIM-V (Invitrogene) and plated at a cellular density of 2.5×105 cells/well in a 96 sterile tissue-culture plate (Nunclon Delta). Serial dilution of wild type group 1 mite polypeptide and group 1 mite polypeptide variant allergens were made up in growth media and added to the cells, together with a media-only control. The plates were then incubated for 7 days at 37 degree C., 5% CO₂, 100% humidity. At the end of the incubation, T cell proliferation was measured by the incorporation of 3[H]-thymidine. 20 hour prior to harvest, 3[H]-thymidine (0.5 μCi) per well was added. The cells were harvested onto glass fiber filters, and 3[H]-thymidine incorporation was measured in scintillate counter. Proliferation was expressed as mean counts per miute (cpm) of 3[H]-thymidine incorporation of triplicate or duplicate wells. The stimulation index (SI) was calculated as the quotient of the cpm obtained by allergen stimulation and the unstimulated control (media-only control).

SI is shown for each donor and a selection of group 1 mite polypeptide variants in table 4. Donor 1-23 represents the 23 dust mite allergic patients, whereas donor 24-26 represents the 3 negative donors.

TABLE 4
Stimulation indexes for T cell proliferation in response to stimulation with group 1 mite
polypeptide and group 1 mite polypeptide variants. Unless otherwise stated in the table,
analysis of T cell proliferation was carried out on stimulation with 1.0 μg/ml group 1 mite
polypeptide or group 1 mite polypeptide variants.

| | | Rec-proDer 1 | | | | | | DP026 | |
|---|---|---|---|---|---|---|---|---|---|
| | nDer p 1 | (0.64 μg/ml) | Rec-Derp1 | DP015 | DP019 | DP024 | DP025 | (0.44 μg/ml) | DP033 |
| Donor 1 | 8.9 | 4.0 | 5.0 | 3.9 | 6.6 | 1.9 | 2.8 | 2.8 | 2.7 |
| Donor 2 | 3.0 | 1.5 | 1.6 | 0.7 | n.d^{a} | 0.7 | 0.2 | 1.8 | 1.4 |
| Donor 3 | 7.1 | 5.3 | 5.6 | 3.7 | 4.8 | 0.7 | 1.0 | 7.8 | 4.8 |
| Donor 4 | 10.4 | 5.3 | 6.7 | 1.1 | n.d | 2.1 | 0.5 | 3.9 | 1.5 |
| Donor 5 | 27.8 | 1.8 | 43.8 | 1.0 | 18.2 | 9.7 | 1.7 | 26.5 | 4.0 |
| Donor 6 | 1.3 | 3.4 | 1.5 | 2.9 | 0.6 | 0.6 | 0.2 | 2.7 | 5.5 |
| Donor 7 | 41.3 | 19.5 | 19.8 | 2.7 | n.d | 5.4 | 1.0 | 7.0 | 7.6 |
| Donor 8 | 1.8 | 0.9 | 0.6 | 0.5 | 0.7 | 0.5 | 0.2 | 0.5 | 0.5 |
| Donor 9 | 13.7 | 5.2 | 9.0 | 1.1 | 8.7 | 1.9 | 0.1 | 9.4 | 2.7 |
| Donor 10 | 35.9 | 25.8 | 25.2 | 3.4 | 30.7 | 15.3 | 1.7 | 13.2 | 48.3 |
| Donor 11 | 4.3 | 1.3 | 8.8 | 0.9 | 11.1 | 1.9 | 0.7 | 2.3 | 1.2 |
| Donor 12 | 12.6 | 8.1 | 8.5 | 1.3 | n.d | 9.8 | 2.3 | 14.1 | 5.2 |
| Donor 13 | 30.9 | 41.1 | 34.0 | 1.6 | n.d | 8.1 | 1.0 | 33.5 | 14.3 |
| Donor 14 | 42.7 | 9.0 | 13.3 | 5.3 | 14.9 | 5.9 | 0.7 | 9.4 | 7.8 |
| Donor 15 | 6.6 | 5.9 | 6.1 | 1.3 | 9.9 | 5.1 | 1.5 | 4.0 | 2.8 |
| Donor 16 | 7.0 | 3.1 | 6.1 | 1.7 | n.d | 1.6 | 0.3 | 6.0 | 3.4 |
| Donor 17 | 3.2 | 2.4 | 3.8 | 6.6 | 5.1 | 2.9 | 0.6 | 3.1 | 6.1 |
| Donor 18 | 15.8 | 3.1 | 4.5 | 3.0 | n.d | 1.6 | 0.5 | 3.5 | 3.2 |
| Donor 19 | 12.2 | 8.6 | 33.7 | 9.8 | 29.2 | 9.1 | 2.1 | 23.7 | 6.3 |
| Donor 20 | 2.6 | 1.3 | 1.6 | 1.2 | n.d | 0.4 | n.d | n.d | 1.2 |
| Donor 21 | 2.3 | 1.9 | 1.9 | 1.5 | n.d | 0.3 | 0.04 | n.d | 2.6 |
| Donor 22 | 8.8 | 5.0 | 5.5 | 3.1 | 7.3 | 2.7 | 0.5 | 8.0 | 4.0 |
| Donor 23 | 3.6 | 3.3 | 3.7 | 2.9 | 2.6 | 1.1 | 0.05 | 3.8 | 2.1 |
| Donor 24 | 1.1 | 2.7 | 2.0 | 0.7 | n.d | 1.0 | n.d | n.d | 1.7 |
| Donor 25 | 1.0 | 1.9 | 1.5 | 1.6 | 1.4 | 0.7 | 0.2 | 1.6 | 1.2 |
| Donor 26 | 3.1 | 6.7 | 22.5 | 0.8 | 5.4 | 3.3 | 0.3 | 6.7 | 3.1 |

^{a}n.d. not determined

The data disclosed in the table above shows that group 1 mite polypeptide variants were able to induce proliferation in primary T cells from dust mite allergic patients. This suggests that the variants can initiate the cellular immune response necessary for antibody production.

### Example 11

### Epitope Mapping Based on Human Anti-Der p 1 Antiserum

Preparation of human IgE beads with specificity for Der p 1 for targeting in selection experiments (anti-Der p 1 beads).

Rabbit anti-human IgE (anti-hIgE) antibodies were covalently linked to commercially available tosyl-activated paramagnetic beads. After inactivation of the remaining linkage sites and washing of the beads according to manufacturer's specification, the anti-hIgE-beads were incubated overnight at 4° C. with pooled sera from patients sensitized to Der p 1 (the sera were 3× diluted in dilution buffer (PBS at pH 6.6)). 200 microliter beads were washed 3× with 40 ml of washing buffer (PBS at pH6.6 plus 0.05% Tween20), then incubated with PBS supplemented with 2% skim milk for an hour at room temperature and washed 3× as before.

Selection of epitope mimics from commercially available phage-displayed peptide libraries: Epitope mimicking peptides were isolated from commercially available phage display libraries of either 7mer, constrained 7mer or 12mer peptide libraries (New England Biolabs). The anti-Der p 1-beads were incubated with 2*10¹¹ library phages for 4 h at room temperature, after which unbound phages were removed by extensive washing. To avoid the enrichment of peptides that were bound to either plain beads or to the anti-hIgE antibody, a specific elution procedure was implemented: After washing, beads were first incubated with PBS supplemented with 0.5% skim milk for an hour at room temperature. After this additional washing step, phages were eluted from the beads by incubation with 25 microM purified Der p 1 in PBS supplemented with 0.5% skim milk, again lasting an hour at room temperature. Only phages in the supernatant of this elution were propagated further. Selected phages were amplified according to the guidelines of the library manufacturer (NEB user manual) after a first round of selection using ER2738 cells. After a second round, cells were infected and spread out for isolation of phages, which were subsequently tested for binding and sequenced.

Phage ELISA to Test Binding to Serum IgE:

100 ng of anti-hIgE antibody in 100 microliter coating buffer (50 mM sodiumcarbonate at pH 9.0) were coated overnight at 4 degree C. on 96-well plates. Unoccupied binding sites were blocked for 2 hrs at room temperature with skimmed milk (2 (wt/vol) % in washing buffer (PBS at pH6.6 plus 0.05% Tween20). Human IgE are then selectively immobilized from serum samples that were diluted 50× in dilution buffer (PBS at pH 6.6) to 100 microliter and incubated for 2 hr at room temperature. Phage preparations from isolated cells infected with 2^{nd} round elution, which contained phages displaying putative epitope mimics, were sampled in varying dilutions for their binding. Bound phages were detected either with mouse anti M13-phage antibody-horseradish peroxidase (HRP) conjugates or with a mouse anti-pill Antibody, Sheep anti-mouse IgG antibody-HRP sandwich when cross-reactivity to the serum in absence of any phage preparation was detected.

1. A variant of a parent group 1 mite polypeptide, wherein the mature polypeptide of the variant comprise one or more mutations in the parent polypeptide in the positions or corresponding to the positions consisting of A10, A12, E13, G29, G30, G32, A46, Y47, S54, L55, D64, A66, S67, G73, T75, I80, Q84, N86, G87, S92, Y93, Y96, A98, R99, E100, Q101, R104, R105, P106, Q109, R110, F11, G112, I113, A132, I144, K145, D146, D148, R151, I158, I159, Q160, R161, D162, N163, G164, Y165, Q166, N179, A180, G182, V183, D184, A205, I208 of SEQ ID NO: 1 or 10, 12, 13, 29, 30, 32, 46, 47, 54, 55, 64, 66, 67, 73, 75, I80, 84, 86, 87, 92, Y93, 96, 98, 99, 100, 101, 104, 105, 106, 109, 110, 111, 112, 113, 132, 144, 145, 146, 148, 151, 158, 159, 160, 161, 162, 163, 164, 165, 166, 179, 180, 182, 183, 184, 205, 208 of the mature der p 1 polypeptide.

2. The variant of claim 1 comprising one or more mutations in the parent polypeptide in the positions or corresponding to the positions consisting of A10, A12, G29, G30, G32, A46, Y47, S54, L55, D64, A66, G73, T75, I80, Q84, N86, G87, S92, Y93, Y96, A98, E100, Q101, R104, R105, P106, R110, F111, G112, I113, A132, I144, K145, D146, I158, I159, Q160, D162, N163, G164, Y165, Q166, N179, A180, G182, V183, D184, A205, I208 of SEQ ID NO: 1 or 10, 12, 29, 30, 32, 46, 47, 54, 55, 64, 66, 73, 75, I80, 84, 86, 87, 92, Y93, 96, 98, 100, 101, 104, 105, 106, 110, 111, 112, 113, 132, 144, 145, 146, 158, 159, 160, 162, 163, 164, 165, 166, 179, 180, 182, 183, 184, 205, 208 of the mature Der p 1 polypeptide.

3. The variant of claim 1 comprising one or more mutations in the parent polypeptide in the positions or corresponding to the positions consisting of A10, A12, G29, G30, G32, A46, S54, L55, D64, A66, G73, T75, Q84, N86, G87, S92, Y96, E100, Q101, R104, R105, P106, R110, I113, A132, I144, K145, D146, D162, N163, G164, Y165, N179, A180, G182, V183, D184 and A205 of SEQ ID NO: 1 or 10, 12, 29, 30, 32, 46, 54, 55, 64, 66, 73, 75, 84, 86, 87, 92, 96, 100, 101, 104, 105, 106, 110, 113, 132, 144, 145, 146, 162, 163, 164, 165, 179, 180, 182, 183, 184 and 205 of the mature Der p 1 polypeptide.

4. The variant of claim 1 having an altered IgE-antigenicity as compared to the parent group 1 mite polypeptide.

5. The variant of claim 4, said variant having at least the same T-cell stimulatory effect compared to the parent group 1 mite polypeptide.

6. The variant of claim 1, said variant inducing an altered immunogenic response in exposed animals, including humans, as compared to the parent group 1 mite polypeptide.

7. The variant of claim 1, said variant inducing an altered immunogenic response in humans, as compared to the parent group 1 mite polypeptide.

8. The variant of claim 1 comprising one or more mutations in the positions or corresponding to the positions consisting of A10, A12, G30, G32, A46, Y47, S54, L55, D64, A66, S67, G87, S92, A98, R99, E100, Q101, R105, R110, F111, G112, I113, I144, K145, D146, D148, R151, 1159, Q160, R161, D162, N163, G164, Y165, Q166, N179, A180, G182, V183, D184, A205, I208 of SEQ ID NO: 1 or 10, 12, 30, 32, 46, 47, 54, 55, 64, 66, 67, 87, 92, 98, 99, 100, 101, 105, 110, 111, 112, 113, 144, 145, 146, 148, 151, 159, 160, 161, 162, 163, 164, 165, 166, 179, 180, 182, 183, 184, 205, 208 of the mature Der p 1 polypeptide.

9. The variant of claim 1 comprising one or more mutations in the positions or corresponding to the positions consisting of A10, A12, G32, S54, L55, A66, S67, G87, A98, R99, F111, G112, I113, I144, D146, D148, I159, R161, G164, Q166, A180, D184, A205 and I208 of SEQ ID NO: 1 or 10, 12, 32, 54, 55, 66, 67, 87, 98, 99, 111, 112, 113, 144, 146, 148, 159, 161, 164, 166, 180, 184, 205 and 208 of the mature Der p 1 polypeptide.

10. The variant of claim 1 comprising a mutation selected from the group consisting of
10 substituted by a residue selected from the group consisting of V, Y, Q, N, E and D;
12 substituted by a residue selected from the group consisting of V, Y, Q, N and F;
32 substituted by a residue selected from the group consisting of V, Y, E, D, N and Q;
54 substituted by a residue selected from the group consisting of N, A, T, V, and Q;
55 substituted by a residue selected from the group consisting of V, N, and Q;
66 substituted by a residue selected from the group consisting of V, H, Y, D, E, N, and Q;
67 substituted by a residue selected from the group consisting of V, H, Y, D, E, N and Q;
87 substituted by a residue selected from the group consisting of V, Y, D and E;
98 substituted by a residue selected from the group consisting of V, N, Q, D or E;
99 substituted by a residue selected from the group consisting of H, Y, V, N, Q, E and D;
111 substituted by a residue selected from the group consisting of V, H, I, and W;
112 substituted by a residue selected from the group consisting of V, H, N, Q, E, D and Y;
113 substituted by a residue selected from the group consisting of V, H, N, Q, E, D and Y;
144 substituted by a residue selected from the group consisting of A, G, Y, N, Q or V;
146 substituted by a residue selected from the group consisting of Y, H, V, I, L, N and Q;
148 substituted by a residue selected from the group consisting of Y, V, I and L;
159 substituted by a residue selected from the group consisting of V, Y, A and G;
161 substituted by a residue selected from the group consisting of A, G, V and Y;
164 substituted by a residue selected from the group consisting of V, H and W;
166 substituted by a residue selected from the group consisting of S, W, Y and F;
180 substituted by a residue selected from the group consisting of V, N, Q and Y;
184 substituted by a residue selected from the group consisting of V, M and Y;
205 substituted by a residue selected from the group consisting of V, W and H;
208 substituted by a residue selected from the group consisting of A, G, V, W and H;
wherein each position corresponds to the position of the mature Der p1 polypeptide.

11. The variant of claim 10 comprising a mutation selected from the group consisting of
A10 substituted by a residue selected from the group consisting of V, Y, Q, N, E and D;
A12 substituted by a residue selected from the group consisting of V, Y, Q, N and F;
G32 substituted by a residue selected from the group consisting of V, Y, E, D, N and Q;
L55 substituted by a residue selected from the group consisting of V, N and Q;
S54 substituted by a residue selected from the group consisting of N, A, T, V, and Q;
A66 substituted by a residue selected from the group consisting of V, H, Y, D, E, N and Q;
S67 substituted by a residue selected from the group consisting of V, H, Y, D, E, N and Q;
G87 substituted by a residue selected from the group consisting of V, Y, D and E;
A98 substituted by a residue selected from the group consisting of V, N, Q, D or E
R99 substituted by a residue selected from the group consisting of H, Y, V, N, Q, E and D;
F111 substituted by a residue selected from the group consisting of V, H, I and W;
G112 substituted by a residue selected from the group consisting of V, H, N, Q, E, D and Y;
I113 substituted by a residue selected from the group consisting of V, H, N, Q, E, D and Y;
I144 substituted by a residue selected from the group consisting of A, G, Y, N, Q or V;
D146 substituted by a residue selected from the group consisting of Y, H, V, I, L, N and Q;
D148 substituted by a residue selected from the group consisting of Y, V, I and L;
I159 substituted by a residue selected from the group consisting of V, Y, A and G;
R161 substituted by a residue selected from the group consisting of A, G, V and Y;
G164 substituted by a residue selected from the group consisting of V, H and W;
Q166 substituted by a residue selected from the group consisting of S, W, Y and F;
A180 substituted by a residue selected from the group consisting of V, N, Q and Y;
D184 substituted by a residue selected from the group consisting of V, M and Y;
A205 substituted by a residue selected from the group consisting of V, W and H;
I208 substituted by a residue selected from the group consisting of A, G, V, W and H;
wherein each position corresponds to the position of SEQ ID NO: 1.

12. The variant of claim 1, wherein the parent group 1 mite polypeptide have in its mature form a sequence which has at least 80% identity to SEQ ID NO: 1.

13. The variant of claim 1, wherein the parent group 1 mite polypeptide have in its mature form a sequence which has at least 80% identity to SEQ ID NO: 2.

14. The variant of claim 1, wherein the parent group 1 mite polypeptide have in its mature form a sequence which has at least 80% identity to SEQ ID NO: 3.

15. The variant of claim 1, wherein the parent group 1 mite polypeptide have in its mature form a sequence which has at least 80% identity to Der m1.

16. The variant of claim 1, wherein the parent group 1 mite polypeptide have in its mature form a sequence which has at least 80% identity to SEQ ID NO: 5.

17. The variant of claim 6, wherein the mutation of the parent group 1 mite polypeptide comprise substitution of an amino acid of one size to an amino acid of a different size, an amino acid of one hydrophilicity to an amino acid of a different hydrophilicity, an amino acid of one polarity to an amino acid of a different polarity or an amino acid of one acidity to an amino acid of a different acidity.

18. The variant of claim 1, wherein mutation in the parent group 1 mite polypeptide comprise insertion of one or more attachment groups for conjugating a polymer.

19. The variant of claim 1, wherein the mutation of the parent group 1 mite polypeptide comprise insertion of one or more additional glycosylation sites.

20. The variant of claim 1, wherein the parent group 1 mite polypeptide is a native group 1 mite polypeptide.

21. A composition comprising a variant of claim 1 and a pharmaceutically acceptable carrier or an adjuvant or a composition thereof.

22. The composition of claim 21, wherein the carrier or adjuvant is selected from saline, glycerol, aluminium hydroxide, aluminium phosphate, calcium phosphate, saponins, squalene based emulsions, monophosphoryl lipid A, synthetic mimics of monophosphoryl lipid A, polylactide co-glycolid (PLG) particles, ISCOMS, liposomes, chitosan, bacterial DNA.

23. A nucleotide sequence encoding the variant of claim 1.

24. A nucleotide construct comprising the nucleotide sequence of claim 23, operably linked to one or more control sequences that direct the production of the variant in a host cell.

25. A recombinant expression vector comprising the nucleotide construct of claim 24.

26. A recombinant host cell comprising the nucleotide construct of claim 24.

27. A method of preparing a variant comprising:
(a) cultivating the recombinant host cell of claim 26 under conditions conducive for production of the variant and
(b) recovering the variant.


SEQUENCE LISTING (5 sequences)

SEQ ID NO: 1 - protein, 320 aa, Dermatophagoides pteronyssinus
  SIGNAL (1)..(18)
  PROPEP (19)..(98)
  mat_peptide (99)..(320)
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 2 - protein, 321 aa, Euroglyphus maynei
  PROPEP (1)..(98): Propeptide containing a signal peptide
MKIILAIASLLVLSAVYARPASIKTFEEFKKAFNKTYATPEKEEVARKNFLESLKYVESNKGAINHLSDLSLDEFKNQFLMNANAFEQLKTQFDLNAETYACSINSVSLPSELDLRSLRTVTPIRMQGGCGSCWAFSGVASTESAYLAYRNMSLDLAEQELVDCASQNGCHGDTIPRGIEYIQQNGVVQEHYYPYVAREQSCHRPNAQRYGLKNYCQISPPDSNKIRQALTQTHTAVAVIIGIKDLNAFRHYDGRTIMQHDNGYQPNYHAVNIVGYGNTQGVDYWIVRNSWDTTWGDNGYGYFAANINLMMIEQYPYVVML

SEQ ID NO: 3 - protein, 321 aa, Dermatophagoides farinae
  PROPEP (1)..(98): Propeptoide containing a signal peptide
MKFVLAIASLLVLSTVYARPASIKTFEEFKKAFNKNYATVEEEEVARKNFLESLKYVEANKGAINHLSDLSLDEFKNRYLMSAEAFEQLKTQFDLNAETSACRINSVNVPSELDLRSLRTVTPIRMQGGCGSCWAFSGVAATESAYLAYRNTSLDLSEQELVDCASQHGCHGDTIPRGIEYIQQNGVVEERSYPYVAREQRCRRPNSQHYGISNYCQIYPPDVKQIREALTQTHTAIAVIIGIKDLRAFQHYDGRTIIQHDNGYQPNYHAVNIVGYGSTQGDDYWIVRNSWDTTWGDSGYGYFQAGNNLMMIEQYPYVVIM

SEQ ID NO: 4 - protein, 30 aa, Dermatophagoides microceras
  MISC_FEATURE (1)..(30): N-terminal fragment
TQACRINSGNVPSELDLRSLRTVTPIRMQG

SEQ ID NO: 5 - protein, 221 aa, Blomia tropicalis
  mat_peptide (1)..(221)
IPANFDWRQKTHVNPIRNQGGCGSCWAFAASSVAETLYAIHRHQNIILSEQELLDCTYHLYDPTYKCHGCQSGMSPEAFKYMKQKGLLEESHYPYKMKLNQCQANARGTRYHVSSYNSLRYRAGDQEIQAAIMNHGPVVIYIHGTEAHFRNLRKGILRGAGYNDAQIDHAVVLVGWGTQNGIDYWIVRTSWGTQWGDAGYGFVERHHNSLGINNYPIYASL